(19) United States
(12) Patent Application Publication
(10) Pub. No.: US 2023/0057033 A1
(43) Pub. Date: Feb. 23, 2023

(54) FLUOROGENIC BETA-LACTAMASE SUBSTRATE AND ASSOCIATED DETECTION METHOD

(71) Applicants: MOLSID, Lyon (FR); ECOLE NORMALE SUPERIEURE DE LYON, Lyon (FR); UNIVERSITE CLAUDE BERNARD LYON 1, Villeurbanne (FR); CENTRE NATIONAL DE LA RECHERCHE SCIENTIFIQUE (CNRS, Paris (FR)

(72) Inventors: Jens HASSERODT, Sainte Foy Les Lyon (FR); Mathieu BORDY, Lyon (FR); Quentin GLENADEL, Clermont Soubiran (FR); Charlie VERRIER, Lyon (FR)

(73) Assignees: MOLSID, Lyon (FR); ECOLE NORMALE SUPERIEURE DE LYON, Lyon (FR); UNIVERSITE CLAUDE BERNARD LYON 1, Villeurbanne (FR); CENTRE NATIONAL DE LA RECHERCHE SCIENTIFIQUE (CNRS), Paris (FR)

(21) Appl. No.: 17/780,855

(22) PCT Filed: Nov. 30, 2020
(86) PCT No.: PCT/EP2020/083976

§ 371 Date: May 27, 2022

(30) Foreign Application Priority Data
Nov. 29, 2019 (EP) 19306540.6

(51) Int. Cl. G01N 33/542; G01N 33/58; C07D 477/24; C07D 501/34; C09B 57/00; C09B 67/44

(52) U.S. Cl. CPC G01N 33/542; G01N 33/582; C07D 477/24; C07D 501/34; C09B 57/00; C09B 67/0083; G01N 2333/986

(57) ABSTRACT

Probes for the detection of β-lactamase-type enzymatic activity. In particular, novel fluorogenic substrates for detecting the presence of a catalytically active β-lactamase and a detection method using such substrates.

### TECHNICAL FIELD

This invention relates to probes for the detection of β-lactamase-type enzymatic activity. In particular, the invention relates to novel fluorogenic substrates for detecting the presence of a catalytically active β-lactamase and a detection method using such substrates.

### BACKGROUND ART

β-lactam antibiotics are a well-known class of antibiotics containing a β-lactam ring in their structure. However, bacteria tend to develop resistance to these antibiotics by producing an enzyme, a β-lactamase, which facilitates the hydrolysis of the antibiotic's β-lactam ring, thus rendering the drug ineffective. Bacterial resistance is becoming a worldwide problem. Among antibiotics-resistant bacteria, those that develop resistance towards carbapenems are currently regarded as untreatable. Indeed, in hospitals, patients harbouring them have a 50% chance of dying of the infection.

In this context, it is therefore useful to detect β-lactamase activity, and in particular carbapenemase activity, in order to identify bacteria which are resistant to β-lactam antibiotics.

The detection of this activity by the capture of fluorescent light issued by a probe would be a much more sensitive method than the collection of white light remnants during a simple absorption by the probe, that is, the detection threshold is much lower. Detection of a fluorescence emission is very easy to implement, so that fluorescent molecules are very attractive tools for the life sciences. For example, the class of fluorophores leading to an intramolecular proton transfer in an excited state, called ESIPT (for “Excited State Intramolecular Proton Transfer”), is described, specifically, in a) Ormson, S. M., et al. Progress in Reaction Kinetics (1994) 19, 45-91; b) Legourrierec, D., et al. Progress in Reaction Kinetics (1994), 19, 211-275; and c) Zhao, 1., Ji, S., Chen, Y., Guo, H., & Yang, P. (2012). Excited state intramolecular proton transfer (ESIPT): from principal photo physics to the development of new chromophores and applications in fluorescent molecular probes and luminescent materials. Physical Chemistry Chemical Physics, 14 (25), 8803. The first interpretation of the elevated fluorescence found in certain phenolic compounds as being an ESIPT phenomenon can be attributed to Weller (for methyl salicylate: Weller, A. (1961). Fast Reactions of Excited Molecules. Progress in Reaction Kinetics and Mechanism 1, 187), and to Heller and Williams (for hydroxyphenyl benzoxazoles: Heller A., et Williams, D. L., 1. Phys. Chem. (1970) 74, 4473-4480).

The class of ESIPT fluorophores is especially attractive to the researcher in the life sciences, due to its exceptional properties in comparison with the conventional fluorophores. The exceptional properties of the ESIPT fluorophores are:

(a) a large Stokes shift often exceeding 130 nm and capable of reaching values of 250 nm which makes instrumental choices possible that maximize the sensitivity of detection; this by escaping/ignoring the auto-fluorescence by cellular and tissular components;

(b) the ability to design fluorophores that emit a brilliant fluorescence in the solid state, a property that is rare among all known fluorophores. This last characteristic makes it possible to produce a high-intensity signal at the activation site, with minimum dilution caused by diffusion;

(c) the ability to design ESIPT fluorophores which issue in the red, or nearly infrared (600 to 850 nm) where tissue transparency is the greatest; a probe using such fluorophores would also be especially suited for imaging in a living animal; and finally,

(d) the ability to design a substrate not issuing fluorescence by replacing the hydrogen atom borne by the hydroxyl of an ESIPT fluorophore with a substitute which has a specific reactivity in relation to a chemical or biochemical analyte, the cleavage of this substitute driving the appearance of the fluorescence.

The sensitivity level of a detection method for enzymatic activity, by use of a substrate resulting in a production of fluorescence, is closely linked (i) to the rate of photo bleaching, (ii) to the degree of accumulation of the fluorescent signal at its production site (and, therefore, to the diffusion rate from this site, and to the question of knowing if the fluorophore precipitates or not) (iii) to the actual extinguishing/lighting mode according to which the substrate functions (lack of background which would be due to a fluorescence of untransformed substrate), and (iv) to the degree of excitation spectrum and emission spectrum stacking (their separation at the baseline being the most favorable configuration; see point a) above). Point (iv) is of a very specific importance, because complete separation at the baseline provides the opportunity of a very broad choice of filters for the light source (in order to excite the fluorophore at every possible wavelength), but even more importantly, for the detector (in order to harvest photons coming from all of the wavelengths issued by the fluorophore). Point (iv) also minimizes disturbance of the detection process by tissue auto-fluorescence (characterized by a weak Stokes shift of natural fluorophores), a recurring problem encountered with established fluorophores, which themselves show mostly a weak Stokes shift.

Among the important class of ESIPT fluorophores, dichloro-HPQ (6-chloro-2-(5-chloro-2-hydroxyphenyl)-4(3H)-quinazolinone; CAS number: 28683-92-3) is especially interesting, given that it is completely insoluble in aqueous/physiological media, while also being highly fluorescent in the solid state and only in the solid state, not in solution.

Several probes for detecting β-lactamase have been developed. However, these probes are either very expensive or do not guarantee a high degree of accumulation of the fluorescent signal at its production site. In practice, genotypical detection (PCR of beta-lactamase messenger RNA) appears to be currently favored by the diagnostics industry in order to detect bacterial resistance, but should be criticized for the high costs and long analysis times it incurs.

In this context, it would be useful to provide improved probes capable of detecting β-lactamase activity, and in particular carbapenemase activity.

One of the objectives of the present invention is to propose novel β-lactamase substrates which are stable in aqueous media and which remain non-fluorescent or mildly fluorescent at a wavelength that is very different from that at which the released fluorophore is itself fluorescent, but which react rapidly with β-lactamase in order to fragment into follow-up molecules, including a small fluorescent molecule.

### SUMMARY

The invention first concerns a compound of formula (I):

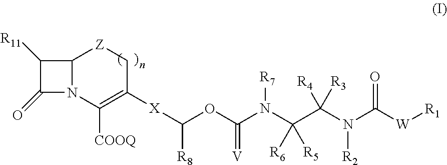

in which:
W is —O— or —NR₁₃—, with R₁₃ being a C₁-C₄ alkyl or a hydrogen atom;
R₁ is such that HWR₁, obtained after cleavage of the —C(O)—WR₁ bond present in formula (I), belongs to the class of fluorophores, preferably to the class of fluorophores leading to an intramolecular proton transfer in an excited state;
R₂, R₃ and R₄ are defined as follows:
   either R₂ is a C₁-C₄ alkyl, R₃ is a C₁-C₄ alkyl or a hydrogen atom, and R₄ is a C₁-C₄ alkyl;
   or R₃ is a C₁-C₄ alkyl or a hydrogen atom and R₂ and R₄ are bonded to each other and form a —(CH₂)ₚ—Y_{q}—(CH₂)ᵣ— chain in direction of R₂ toward R₄, wherein
      Y is O, NR₁₄, N(R₁₄)₂⁺ or S, preferably NR₁₄ or N(R₁₄)₂⁺,
      p=0, 1, 2, 3, 4 or 5,
      q=0 or 1,
      r=0, 1, 2, 3, 4 or 5,
      p+q+r=4, 5, or 6, and
      each R₁₄ represents independently a hydrogen atom, a C₁-C₆ alkyl, an amino protecting group, or -(L)_{q}-GP, with q which is equal to 0 or 1, L which is a linking arm and GP which is a hydro solubilizing group;
   or R₂ is a C₁-C₄ alkyl and R₃ and R₄ are bonded together and form, with the carbon atom to which they are bonded, an aliphatic carbocycle;
R₅ and R₆ are identical or different and represent, independently of each other, a hydrogen atom, a C₁-C₄ alkyl, or a C₅-C₁₀ aryl;
R₇ is a hydrogen atom, or a group selected from C₁-C₄ alkyl and C₁-C₄ alkoxy;
R₈ represents a hydrogen atom;
V represents an oxygen atom or a sulfur atom;
Z is —S—, —SO— or —CR₉R₁₀—; with R₉ and R₁₀ being identical or different and representing, independently of each other, a hydrogen atom or a C₁-C₄ alkyl;
Q is H, a cation or R16, where R16 is selected from C₁-C₆ alkyl optionally substituted with an aryl or a O—(CO)—R, with R being independently selected from H, C₁-C₆ alkyl and C₃-C₆ cycloalkyl; and
R₁₁ is an organyl group, an organylamino group, an organyloxy group, or an organylthio group,
X represents a bond, or a group selected from

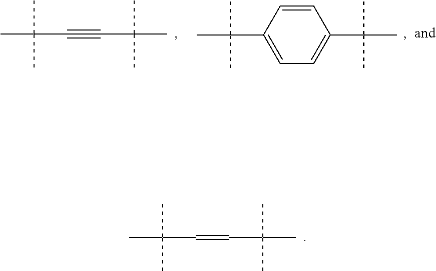

Applicants have developed a line of β-lactamase substrates that have the following properties:
rapid response to the activity of members of the extended-spectrum β-lactamases (ESBLs) or to that of a carbapenemase by generating intense fluorescence;
release of a fluorophore, preferably an ESIPT fluorophore, upon enzyme conversion, including the “ELF97 alcohol” with its recognized unique properties of signal retention at the site of enzyme activity;

and
largely or fully non-fluorescent in the absence of the target enzyme activity, thus maximizing detection sensitivity via an advantageous signal-to-background ratio.

Thus, the compounds (I) according to the invention reveal the presence of β-lactamase activity by the generation of fluorescence.

More specifically, the probe is invisible before encountering the targeted β-lactamase enzyme, and may thus be called a “stealth probe”. However when it is chemically modified by said enzyme (hydrolytic opening of the beta-lactam ring), it fragments via a cascade reaction to release a detectable fluorophore. The probe comprises 3 molecular components: i) a self-immolative spacer which bears, at one end, ii) a cephalosporin or carbapenem group playing the role of substrate for the target enzyme and, at the other end iii) a WR1 group which, when released as HWR₁ by said fragmentation, belongs to the class of fluorophores.

The choice of spacer in the present invention furnishes two essential advantages for the corresponding molecular probe: (a) its chemical link with WR1 is stable towards spontaneous hydrolysis and thus the release of the fluorophore and the production of a false positive signal, and (b) its chemical link with the cephalosporin or carbapenem unit is of a carbamate nature which not only ensures hydrolytic stability but is of great importance because the link's small size ensures sufficient molecular recognition by the enzyme and thus an efficient turnover rate.

The two ways of pre-organizing the spacer for cyclization, consisting either of introducing two alkyl substitutes (or forming a carbocyclic ring), on the alpha carbon of the —N—C(V)—O— group, or of including the bond between the group nitrogen —N—C(V)—O— and its alpha carbon in a heterocyclic ring, accelerate the fragmentation process.

This invention therefore concerns the compounds of formula (I), regardless of their implemented variant described in this patent application, for the detection of β-lactamase or carbapenemase activity in in vitro diagnostic tests, including live cell analysis (bacteria). The compounds of formula (I) according to the invention may also be used to detect a β-lactamase, in vivo, in animals. Accordingly, the invention also relates to compounds of formula (I) according to the invention for the in vivo detection, in human beings, of a β-lactamase.

More specifically, the invention concerns a method for detecting, in vitro or ex vivo, the presence of β-lactamase activity comprising the steps of:
putting a sample to be analyzed into contact with a compound (I),
applying suitable conditions in order to make possible the formation of a fluorescent precipitate by cleavage of the covalent bond between C(═V) and NR₇, followed by a cleavage of the —C(O)—WR₁, bond, leading to the release of HWR₁,
quantitative or qualitative analysis of the fluorescent precipitate, and
correlation of the quantitative or qualitative analysis of the fluorescent precipitate to the presence or absence of β-lactamase in the sample.

The precipitate which can be obtained using the compounds of formula (I) according to the invention, by cleavage of the covalent bond between C(═V) and NR_{S}, followed by a cleavage of the —C(O)—WR₁, bond, leading to the release of HWR₁, after a cyclization of the spacer, is strongly fluorescent, while the compound of corresponding formula (I) is mildly fluorescent or not fluorescent at all. The compounds according to the invention are β-lactamase substrates that operate according to an off/on mode and thus allow for the probing of this enzyme activity without the necessity to wash away excess probe before readout (“no-wash assay”). Therefore, the compound according to the invention is not fluorescent or mildly fluorescent, when no enzyme is present (off mode), but in the presence of β-lactamase enzyme, the compound is fragmented and releases a fluorophore which can be detected (on mode).

In particular, the detection method according to the invention can be implemented in physiological conditions, specifically in an aqueous medium buffered to a pH of 7.4.

The invention also concerns a compound of formula (II), intermediate in the synthesis of the compound of formula (I):

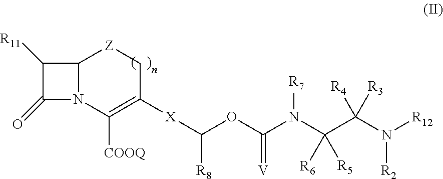

in which:
R₂, R₃, R₄, R₅, R₆, R₇, R₈, R₁₁, Z, n, Q, X and V are as defined for compound (I),
R₁₂ represents a hydrogen atom, or an amine function protecting group.

The invention also concerns a method for the preparation of a compound (I) comprising the following steps:
implementation of a compound (II) as defined herein,
implementation of a compound (III) of formula

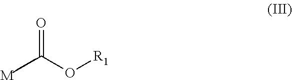

with R₁ as defined for compound (I) and M representing a leaving group, preferably selected from a halide atom, and in particular CI, an imidazolyl group, a triazolyl group, and a para-nitrophenoxyl, and preferentially with M representing a para-nitrophenoxyl.
obtaining compound (I) by addition reaction of said compound (II) to compound (III).

The different compounds according to the invention can be found in all possible optical isomer forms, possibly in the form of a mixture according to all proportions, at least if not otherwise specified. According to a specific embodiment, the compounds according to the invention comprising an asymmetric carbon atom are found in a racemic form, with the R and S forms being found in almost equal proportions. According to another embodiment, the formula (I) compounds of the invention can be found in an isomerically enriched form, either diastereomerically or enantiomerically, with a diastereomeric or enantiomeric excess greater than 80%, or even greater than 95%, or in pure isomeric form, namely with a diastereomeric or enantiomeric excess greater than 99%.

The compounds can be isolated in an enriched form in a diastereomer or enantiomer by classic separation techniques: for example, fractional recrystallizations of a racemic salt with an optically active acid or base for which the principle is well-known or, most often, classic chromatography techniques on the chiral or non-chiral phase.

If applicable, compounds according to the invention may be found in the form of a salt, specifically a hydrochloride, a hydroacetate, a hydrotrifluoroacetate, a sodium salt, or an ammonium salt.

The invention will be described in a more detailed manner. First, certain terms used will be defined.

### DETAILED DESCRIPTION

### Definitions

By “aliphatic heterocycle”, in the context of this invention, is understood a saturated cycle, substituted or not substituted, comprising 3 to 20 members, preferably 5 to 10 members, and more preferably, still, 5, 6, 7 or 8 members, and comprising at least one hetero-atom, such as O, N, or S.

By “aliphatic carbocycle”, in the context of this invention, is understood a saturated cycle, substituted or not substituted, comprising 3 to 30 members, preferably 5 to 10 members, and more preferably still, 5, 6, 7 or 8 members constituted exclusively by carbon atoms.

By “alkyl”, in the context of this invention, is understood a saturated hydrocarbon chain which can be linear or branched. Preferably, the term alkyl designates, at least if not otherwise specified, an alkyl group comprising 1 to 12 carbon atoms and, preferably 1 to 6 carbon atoms, and specifically an alkyl (C₁-C₄) group. Methyl, ethyl, n-propyl, isopropyl, and tert-butyl are examples of (C₁-C₄) alkyl groups (alkyl with 1 to 4 carbon atoms).

By “alkylene” is understood a divalent alkyl group.

By “heteroalkyl”, in the context of this invention, is understood a straight or branched hydrocarbon chain consisting of 1 to 6 carbon atoms, and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group or at the position at which the alkyl group is attached to the remainder of the molecule.

By “aryl” is understood a mono- bi- or polycyclic ring, unsaturated hydrocarbon comprising, at least if not otherwise specified, from 5 to 24 members, from 5 to 20 members, from 5 to 15 members, preferably from alternating simple bonds and double bonds, and comprising at least one aromatic ring. As an example of any aryl group, we can cite the phenyl, naphtyl, anthracenyl, phenanthrenyl and cinnamyl groups. The term aryl also includes such mono-, bi- or polycyclic, unsaturated, hydrocarbon rings for which one of the constituting carbons is found in the —C(O) carboxy form, such as the 1H-phenalene-1-one (CAS no. 548-39-0).

By “arylene” is understood a divalent aryl group.

By “hetero-aryl” is understood a mono-, bi- or polycyclic carbocyclic ring, comprising, at least unless otherwise specified, from 5 to 24 members, preferably from 6 to 20 members, more preferably from 6 to 15 members, and comprising at least one aromatic group and at least one hetero-atom, chosen from among the atoms of oxygen, nitrogen or sulfur, integrated into the carbocyclic ring. By way of example of a hetero-aryl group, we may cite the 2-, 3- or 4-pyridinyl, 2- or 3-furoyl, 2- or 3-thiophenyl, pyrrolyl, imidazolyl, pyrazolyl, thiazolyl, benzimidazolyl, bensothiazolyl, oxazolyl, benzoxazolyl, isoxazolyl, pyridinyl, pyrazinyl, pyrimidinyl, tetrazolyl, thiadazolyl, oxadiazolyl, triazolyl, pyridazinyl, indolyl, oxanyl, 4(1H)-quinolinonyl, dibenzothiophenyl, dibenzofuranyl and 9H-carbazolyl. The term heteroaryl also includes said groups for which one of the constituting carbon atoms is found in the carboxy —C(O)— form, such as 4(3H)-pyrimidinonyl, 4(3H)-quinazolinonyl, or 4(1H)-quinolinone.

When it is stated that a group is substituted without further specification, this means that it is substituted by one or several substituents, specifically chosen from among the atoms of chlorine, bromine, iodine or fluorine, the cyano, alkyl, trifluoralkyl, trifluoromethyl, alkenyl, alkynyl, cycloalkyl, aryl, hetero-aryl, heterocyclo-alkyl, amino, alkylamino, diaklyamino, hydroxy, alkoxy, aryloxy, alkoxycarbonyl, aryloxycarbonyl groups, said groups themselves being able to be substituted. The terms used for the definition of these substitutes are those usually recognized by the person skilled in the art.

By “alkoxy” and “aryloxy”, are respectively understood an —O-alkyl and —O-aryl group, with alkyl and aryl as defined in the context of this invention.

By “haloalkyl” is understood a saturated, linear or branched hydrocarbon chain in which at least one hydrogen atom has been replaced by a halogen atom.

By “β-lactamase” is understood a β-lactamase enzyme which has the capacity to catalyze the hydrolysis of β-lactam, so as to open the β-lactam cycle.

By “β-lactam” is understood a four-membered ring cyclic amide.

Classically, the term “alkenyl” designates a hydrocarbon chain, linear or branched, comprising at least one double carbon-carbon bond, and presenting, unless it is otherwise specified, from 2 to 20 carbon atoms, and preferably from 2 to 6 carbon atoms.

In the context of this invention, the term “alkenylene” designates a divalent alkenyl group.

The term “alkynyl” designates a hydrocarbon chain, linear or branched, comprising at least one triple carbon-carbon bond, and presenting, unless it is otherwise specified, from 2 to 12 carbon atoms, and preferably from 2 to 6 carbon atoms.

By “alkynylene” is understood a divalent alkynyl group.

By “linking arm”, is understood a divalent group linking covalently two moieties of the compound.

By “organyl group” is understood any organic substituent, regardless of functional type, having one free valence at a carbon atom, which is used to connect said organyl group to the compound.

By “organylthio group” is understood any organic substituent, regardless of functional type, having one free valence at a sulfur atom, which is used to connect said organyl group to the compound.

By “organyloxy group” is understood any organic substituent, regardless of functional type, having one free valence at an oxygen atom, which is used to connect said organyl group to the compound.

By “organylamino group” is understood any organic substituent, regardless of functional type, having one free valence at a nitrogen atom, which is used to connect said organyl group to the compound.

By “water-solubilizing group” or “hydro solubilizing group” is understood a hydrophilic group which makes it possible to improve the solubility of the probe in an aqueous medium, in relation, specifically, to a probe that only differs from it by the replacement of a water-solubilizing group by a hydrogen atom. In particular, said water-solubilizing group can modify the electrostatic properties of the probe.

As used herein, the terms “protecting group” refer to a chemical substituent which can be selectively removed by readily available reagents which do not attack the regenerated functional group or other functional groups in the molecule. Suitable protecting groups are known in the art and continue to be developed. Suitable protecting groups may be found, for example in Wutz et al. (“Greene's Protective Groups in Organic Synthesis, Fourth Edition,” Wiley-Interscience, 2007). Protecting group for protection of the amino group as described by Wutz et al. (pages 696-927), are used in certain embodiments. Representative examples of amino protecting groups include, but are not limited to, t-butyloxycarbonyl (Boc), 9-fluorenyl methoxycarbonyl (Fmoc), Acetyl (Ac), carboxybenzyl group (Cbz), benzyl group (Bn), allyl, and trifluoroacetyl.

“Fluorescence” is the property by which a molecule that is excited by light of a given wavelength emits light of a longer wavelength. Fluorescence is a phenomenon that results from the interaction of a fluorophore with an incident photon. This process is also called excitation. The absorption of the photon results in an electron in the fluorophore to go from its basic state to a higher energy level. Then, the electron returns to its original level by emitting a photon. This process is called fluorescence emission. The fluorophore then emits light of a longer wavelength than that of the absorbed photon. This is due simply to the fact that the energy of the emitted photon is less than that of the absorbed photon, due to the dissipation of energy during the life span of the excited state. This is the definition given in patent application WO 2004/058787.

The compounds (I) according to the invention are called “β-lactamase substrate” because they are transformed into another substance during a chemical reaction, in particular, a hydrolysis, catalyzed by a β-lactamase. During such a reaction in an aqueous medium, the compounds (I) (also called “probes”) are cleaved under the action of the target β-lactamase, which leads to the fragmentation of the probe, including the formation of a compound that is highly insoluble and precipitates on site; upon adoption of the solid state it begins to fluoresce intensely if excited by light of proper wavelength.

The “spacer” in the context of this invention is the fragment of the compound (I) which bears, at one end, ii) a β-lactam group playing the role of substrate for the target enzyme and, at the other iii) an WR1 group which, when released as HWR₁ by said fragmentation, belongs to the class of fluorophores, more specifically to the class of solid-state fluorophores.

### Compound of Formula (I)

This invention concerns a compound of formula (I):

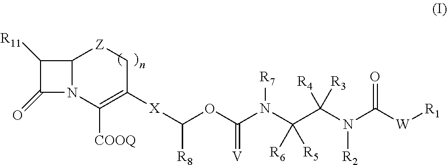

in which:
W is —O— or —NR₁₃—, with R₁₃ being a C₁-C₄ alkyl or a hydrogen atom;
R₁ is such that HWR₁, obtained after cleavage of the —C(O)—WR₁ bond present in formula (I), belongs to the class of fluorophores, preferably to the class of fluorophores leading to an intramolecular proton transfer in an excited state;
R₂, R₃ and R₄ are defined as follows:
   either R₂ is a C₁-C₄ alkyl, R₃ is a C₁-C₄ alkyl or a hydrogen atom, and R₄ is a C₁-C₄ alkyl;
   or R₃ is a C₁-C₄ alkyl or a hydrogen atom and R₂ and R₄ are bonded to each other and form a —(CH₂)ₚ—Y_{q}—(CH₂)ᵣ— chain in direction of R₂ toward R₄, wherein
      Y is O, NR₁₄, N(R₁₄)₂⁺ or S, preferably NR₁₄ or N(R₁₄)₂⁺
      p3 p=0, 1, 2, 3, 4 or 5,
      q=0 or 1,
      r=0, 1, 2, 3, 4 or 5,
      p+q+r=4, 5, or 6, and
      each R₁₄ represents independently a hydrogen atom, a C₁-C₆ alkyl, an amino protecting group, or -(L)_{q}-GP, with q which is equal to 0 or 1, L which is a linking arm and GP which is a hydro solubilizing group;
   or R₂ is a C₁-C₄ alkyl and R₃ and R₄ are bonded together and form, with the carbon atom to which they are bonded, an aliphatic carbocycle;
R₅ and R₆ are identical or different and represent, independently of each other, a hydrogen atom, a C₁-C₄ alkyl, or a C₅-C₁₀ aryl;
R₇ is a hydrogen atom, or a group selected from C₁-C₄ alkyl and C₁-C₄ alkoxy;
R₈ represents a hydrogen atom;
V represents an oxygen atom or a sulfur atom;
n is 0 or 1;
Z is —S—, —SO— or —CR₉R₁₀—; with R₉ and R₁₀ being identical or different and representing, independently of each other, a hydrogen atom or a C₁-C₄ alkyl;
Q is H, a cation or R16, where R16 is selected from C₁-C₆ alkyl optionally substituted with an aryl or a O—(CO)—R, with R being independently selected from H, C₁-C₆ alkyl and C₃-C₆ cycloalkyl; and
R₁₁ is an organyl group, an organylamino group, an organyloxy group, or an organylthio group,
X represents a bond, or a group selected from

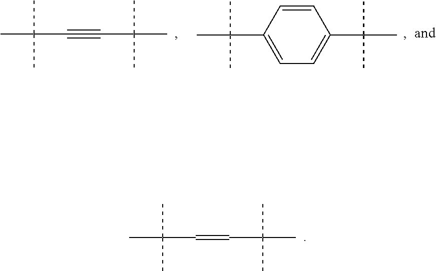

In a specific embodiment, the compound (I) is of formula (Ia):

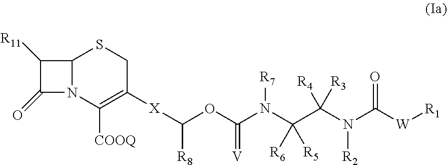

where R₁, R₂, R₃, R₄, R₅, R₆, R₇, R₈, R₁₁, Q, W, X and V are as defined for compound (I).

In another embodiment, the compound (I) is of formula (Ib):

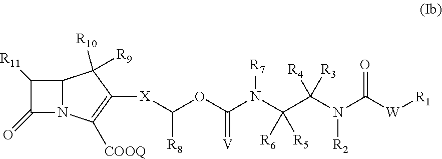

where R₁, R₂, R₃, R₄, R₅, R₆, R₇, R₈, R₉, R₁₀, R₁₁, Q, W, X and V are as defined for compound (I).

Compound of formula (Ib) is particularly useful to detect carbapenemase activity.

The R₁ group is selected so that the obtained fluorescent precipitate which corresponds to HWR₁, released after cleavage of the —C(O)—WR₁ bond, belongs to the class of fluorophores, preferably to the class of fluorophores leading to an intramolecular proton transfer in an excited state (ESIPT).

The ESIPT fluorophores show a Stokes shift which exceeds 100 nm and often approach 200 nm. All ESIPT fluorophores lose this emission of fluorescence corresponding to a Stokes shift greater than 100 nm, if their WH group of the phenolic type gives rise to the intra-molecular transfer of a proton in the excited state, is alkylated, acylated or otherwise functionalized. This functionalization prevents the transfer of a hydrogen atom, during excitation by irradiation, and thus prevents the emission of fluorescence characteristic of the proton transfer method.

The incorporation of the HWR₁ into the carbamate or urea group of the formula (I) compound prevents the proton transfer. The intra-molecular proton transfer may then occur using the group obtained following the scission of the —C(O)—WR₁ bond.

Most often, the R₁ group corresponds to a phenyl group which is non-substituted or substituted and/or which is merged with one or more unsaturated carbocycles, possibly comprising a hetero-atom such as nitrogen. When W is O, the —OR₁ phenoxy derivative, when it is not bonded to the substrate, corresponds in its protonated form to an HO—R₁ phenolic derivative which belongs to the ESIPT class of fluorophores.

We may specifically refer to applications WO 2013/045854, WO 2014/020285, and WO 2015/197981 which give examples of such ESIPT fluorophores which can be used in this invention.

Preferably, R₁ comprises an aromatic group comprising one or more aromatic rings, substituted or non-substituted, said rings being able to comprise one or more hetero-atoms chosen from among the nitrogen, oxygen or sulfur atoms and/or one or more carbon atoms in the form of a C═O carbonyl.

WR₁ can be an aromatic group —OR₁ group according to formula (A1):

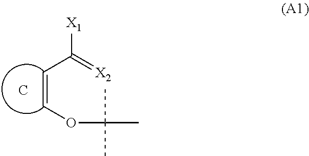

in which:
either X₂ is an oxygen atom and X₁ is a —NH₂, —OH, —SH, C₁-C₂₀ alkyl, C₅-C₂₄ aryl, C₂-C₆ alkenyl, —O-(C₁-C₂₀ alkyl), —O-phenyl, —NH-(C₁-C₂₀ alkyl), —NH-phenyl, —S-(C₁-C₂₀ alkyl) or —S-(C₅-C₂₄ aryl group), said alkyl, aryl, alkenyl and phenyl groups being optionally substituted;

or X₂ represents a nitrogen atom and is bound to X₁ which then represents CH, O, S, N or, NH to form a C₅-C₂₄ heteroaryl, optionally substituted;

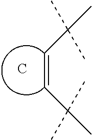

represents a C₅-C₂₄ aryl or a C₅-C₂₄ heteroaryl, optionally substituted, for example, chosen from among the phenyl, naphtyl groups, and:

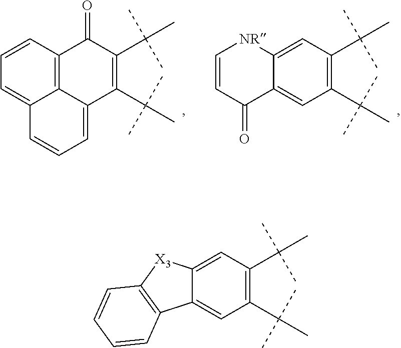

said groups being optionally substituted;

with X₃ which represents S, O or NRd and Rd which represents a hydrogen atom or a C₁-C₄ alkyl group.

Advantageously, —OR₁ is of the aryloxy type and corresponds, preferably, to one of the following preferred structures (A2), (A3) or (A4):

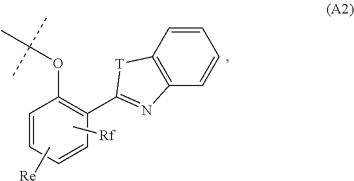

in which:
T is —NH—C(O)—, —S—, —O—, —NH—, —N(C₁-C₂₀ alkyl)- or —N(C₅-C₂₄ aryl)-;
Re is a hydrogen atom or an electron-withdrawing group such as —CN or —COORh, with Rh which represents a C₁-C₄ alkyl group, or Re is —CONRiRj, with Ri and Rj, identical or different, which represent a hydrogen atom, or a C₁-C₄ alkyl group, or Re is —CF₃, a C₂-C₆ alkenyl, or a heteroaryl, said heteroaryl and alkenyl being optionally substituted;
Rf is a hydrogen atom, a chlorine, bromine, iodine or fluorine atom, —OH, —NH₂, —NRkRI, —NHRk or —ORk, with Rk and RI, identical or different, which each, independently, represent a C₁-C₄ alkyl group;
or Re and Rf are bonded to each other to form a hydrocarbon chain comprising 4 or 5 members, saturated or unsaturated, substituted or non-substituted, possibly interrupted by one or more hetero-atoms chosen from among N, S and O; and
Rg is a hydrogen, Br, Cl, I or F atom;

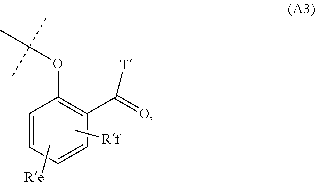

in which:
T′ is —NH₂, —OH, a C₅-C₂₄ aryl group, a C₁-C₄ alkyl group, —SH, —NHR′g, —OR′g, —NR′gRh′, —SR′g, or an optionally substituted C₂-C₆ alkenyl group, or a heteroaryl, R′g and Rh′, identical or different, representing a C-C₄ alkyl or C₅-C₂₄ aryl group;
R′e is a hydrogen atom or an electron-withdrawing group such as —CN or —COOR′i, with R′i which represents a C₁-C₄ alkyl group, or R′e is —CONR′jR′k, with R′j and R′k, identical or different, which represent a hydrogen atom, or a C₁-C₄ alkyl group, or R′e is —CF₃, or a 2-oxazolyl, 2-thiazolyl, 2-imidazolyl, 2-benzo imidazolyl, 4-pyrimidinone-2-yl or quinazolinone-2-yl group;
R′f is a hydrogen, chlorine, bromine, iodine or fluoride atom, —OH, —NH₂, —NR′IR′m or —OR′I, with R′I and R′m, identical or different, which represent a C₁-C₄ alkyl group;
or R′e and R′f are bonded to each other to form a hydrocarbon chain comprising 4 or 5 members, saturated or unsaturated, substituted or non-substituted, possibly interrupted by one or more hetero-atoms chosen from among N, S and O;

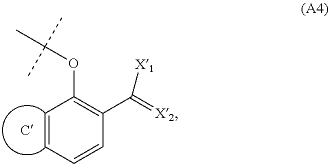

in which
either X^{′}₂ is an oxygen atom and X′₁ is a —NH₂, —OH, —SH, C₁-C₂₀ alkyl, C₅-C₂₄ aryl, C₂-C₆ alkenyl, —O-(C₁-C₂₀ alkyl), —O-phenyl, —NH-(C₁-C₂₀ alkyl), —NH-phenyl, —S-(C₁-C₂₀ alkyl) or —S-(C₅-C₂₄ aryl group), said alkyl, aryl, alkenyl and phenyl groups being optionally substituted;

or X′₂ represents a nitrogen atom and is bound to X₁ which then represents CH, O, S, N or, NH to form a C₅-C₂₄ heteroaryl, optionally substituted;

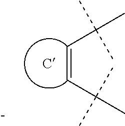

represents a C₅-C₁₀ aryl or a C₅-C₁₀ heteroaryl, optionally substituted.

In the formulas (A1) to (A4), when a substituent is said to be optionally substituted, it is optionally substituted by one or more substituents, preferably selected from: a halide, —CN, C₁-C₆ alkyl, C₁-C₆ haloalkyl, C₂-C₆ alkenyl, C₂-C₆ alkynyl, C₁-C₆ alkoxy, C₅-C₁₀ cycloalkyl, C₅-C₁₀ aryl, C₅-C₁₀ heteroaryl, C₅-C₂₄ heterocycloalkyl, —NH₂, —NH(C₁-C₆ alkyl), —N(C₁-C₆ alkyl)₂, hydroxy, oxo, aryloxy, alkoxycarbonyl, aryloxycarbonyl groups, said cycloalkyl, aryl, heteroaryl, heterocycloalkyl being able to be substituted by a halide, C₁-C₆ alkyl, C₁-C₆ haloalkyl, C₁-C₆ alkoxy, oxo, —NH₂, —NH(C₁-C₆ alkyl), or —N(C₁-C₆ alkyl)₂.

According to a specific embodiment of the invention, WR1 is an aromatic group with —OR1 which responds to one of the following formulas, (A5) or (A6):

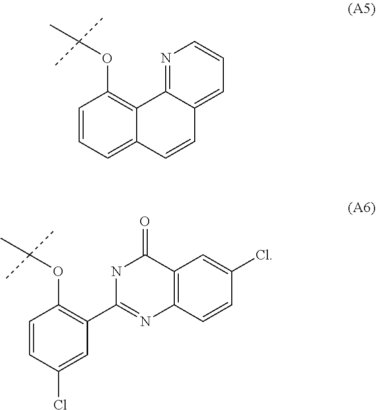

The very large Stokes shift of such fluorophores (approximately 170 nm for A6) or of any analog of the HPQ will contribute to the excellent sensitivity of the probe and render the released fluorophore easily distinguishable from the native fluorescence which may come from the biological sample on which the analysis will be conducted.

According to an embodiment, R1 is selected from the group consisting of fluoresceins (including rhodamines and rhodols), coumarins (including 7-amino- and 7-hydroxy-coumarins), cyanines, phenoxazines and acridinones.

According to one embodiment of the invention, R₂ is a (C₁-C₄) alkyl, R₃ is a (C₁-C₄) alkyl or a hydrogen atom, and R₄ is a (C₁-C₄) alkyl. According to another specific embodiment, R₂, R₃ and R₄, identical or different, represent a (C₁-C₄) alkyl group, for example, methyl or ethyl. According to a specific embodiment, R₂=R₃=R₄=—CH₃.

In another embodiment, R₂ is a C₁-C₄ alkyl and R₃ and R₄ are bonded together and form, with the carbon atom to which they are bonded, an aliphatic carbocycle. For example, R₃ and R₄ are bonded to each other and form a —(CH₂)m— chain with m=3, 4, 5 or 6.

Advantageously, R₃ is a hydrogen atom or a C₁-C₄ alkyl, preferably a hydrogen atom, and R₂ and R₄ are bonded to each other and form a —CH₂CH₂—Y—CH₂—chain in the direction of R₂ toward R₄, Y representing —CH₂—, —NR₁₄—, or —N(R₁₄)₂⁺— with R₁₄ representing a hydrogen atom or -(L)_{q}-GP, with q which is equal to 0 or 1, L which is a linking arm and GP which is a hydro solubilizing group.

In a specific embodiment, R₃ is a C₁-C₄ alkyl or a hydrogen atom and R₂ and R₄ are bonded to each other and form a —(CH₂)ₚ—Y_{q}—(CH₂)ᵣ— chain in direction of R₂ toward R₄, wherein
Y is O NR₁₄, N(R₁₄)₂⁺or S, preferably NR₁₄ or N(R₁₄)₂⁺,
p=0, 1, 2, 3, 4 or 5,
q=0 or 1,
r=1, 2, 3, 4 or 5,
p+q+r=4, 5, or 6, and
each R₁₄ represents independently a hydrogen atom, a C₁-C₆ alkyl, an amino protecting group or -(L)_{q}-GP, with q which is equal to 0 or 1, L which is a linking arm and GP which is a hydro solubilizing group.

For example, R₂ and R₄ are bonded to each other and form a —(CH₂)ₚ—Y_{q}—(CH₂)ᵣ— chain in direction of R₂ toward R₄, wherein
Y is NR₁₄ or N(R₁₄)₂⁺,
p=2, 3, 4 or 5,
q=1,
r=0, 1, 2, 3, 4 or 5,
p+q+r=3, 4, 5, or 6, and
each R₁₄ represents independently a hydrogen atom, a C₁-C₆ alkyl, an amino protecting group or -(L)_{q}-GP, with q which is equal to 0 or 1, L which is a linking arm and GP which is a hydro solubilizing group, or

R₂ and R₄ are bonded to each other and form a —(CH₂)ₚ—Y_{q}—(CH₂)ᵣ— chain in direction of R₂ toward R₄, wherein
p=2, 3, 4 or 5,
q=0,
r=0, 1, 2, 3, 4 or 5, and
p+q+r=4, 5, or 6.

In a specific embodiment, Y is N(R₁₄)₂⁺, the positive charge is on the nitrogen atom, it is therefore an ammonium. In this case, a counter ion is present. The counterion can be selected from the group consisting of halide, trifluoroacetate, and acetate.

In a specific embodiment, q=1 and L is a linking arm and, specifically, a -(L₁)m₁-(L₂)m₂-(L′₁)m′₁-arm (in the piperazine direction->GP group) with:
L₁ and L′₁, identical or different, which are chosen from among —O—, —NH—, —N(C₁-C₆) alkyl)-, —N(phenyl)-, —N(aryl)-, —C(O)—, —C(O)O—, —OC(O)—, —OC(O)—O—, —NHC(O)—O—, —OC(O)—NH—, —NHC(O)—NH—, —S—, —SOC—, —N═N—, —NHC(O)—and —CONH—;
L₂ which is chosen from among the following divalent groups: (C₁-C₂₀)alkylene, (C₁-C₂₀)alkenylene, (C₁-C₂₁)alkynylene, (C₆-C₂₄)arylene, (C₇-C₄₄)alkylarylene, (C₇-C₄₄)alkenylarylene, (C₇-C₄₄)alkynylarylene, (C₇-C₄₄) alkylcycloalkylene, (C₇-C₄₄)alkenylcycloalkylene, (C₇-C₄₄)alkynylcycloalkylene (C₇-C₄₄)alkylheterocycloalkylene, (C₇-C₄₄)alkenylheterocycloalkylene, (C₇-C₄₄)alkynylheterocycloalkylene; said groups being possibly interrupted or terminated by a triazole group, and being able to be substituted or non-substituted, specifically by one or more substitutes chosen from among the (C₁-C₁₀)alkoxy, (C₆-C₁₀)aryl, amide, imide, phosphide, nitride, (C₁-C₁₀)alkenyl, (C₁-C₁₀) alkynyl and —OH; and
m₁, m′₁ and m₂, identical or different, which are equal to 0 or 1.

The L arm, when present, will be chosen to extend the GP group from piperazine or for synthesis reasons. According to one preferred embodiment, L represents -(L₁)m₁-(L₂)m₂-(L′₁)m′₁ with L₁=—C(O)—, m₁=m₂=1, m′₁=1 or 0 and L₂ and L′₁ as defined above, and, specifically, L represents —C(O)—(CH₂)ₚ-L₃- with p which is equal to 1, 2, 3 or 4 and L3 which is a triazole group and specifically a 1H-1,2,3-triazole group.

GP is a water-solubilizing group. As an example of a water-solubilizing group, we cite the groups that can form a charged species in aqueous solution. As an example of water-solubilizing GP group, we cite the
F1 functions chosen from among the amines (primary, secondary, tertiary, or quaternary), am idine, guanidine or tetrazole;
the F2 cationic or anionic functions, and specifically the ammonium, carboxylate, sulfonate or phosphate type groups;
the groups comprising one or more of these F1 and/or F2 functions;
the polyethylene glycols;
the sugars or polysaccharides such as glucose, galactose and mannose;
the peptide groups such as poly-lysine, poly-arginine, the TAT-peptides, and
amino acids.

As an example of amine functions, we cite —NH₂, —NH(C₁-C₄) alkyl, and the dialkylamines in which the alkyl groups are identical or different and comprise 1 to 4 atoms of carbon.

These two ways of pre-organizing the spacer for cyclization, consisting either of introducing two alkyl substitutes (or forming a carbocyclic ring) on the alpha carbon of the —N—C(V)—O— group, or of including the bond between the group nitrogen —N—C(V)—O— and its alpha carbon in a heterocyclic ring, accelerate the immolation process.

According to one embodiment, R₅ and R₆ are identical and represent a hydrogen atom. According to one embodiment, R₇ represents a hydrogen atom or an (C₁-C₄) alyl group such as a methyl, and preferably a hydrogen atom.

According to one embodiment, R₅, R₆ and R₇ each represent a hydrogen atom.

X represents a bond, or a group selected from

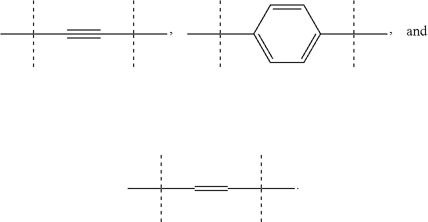

In the present case, the double bond can have any configuration (Z or E). According to an embodiment, X represents a bond, or a group selected from

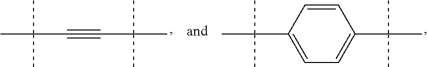

preferably X is a bond.

R₁₁ can be selected from C₁-C₆ alkyl, C₁-C₆ heteroalkyl, C₃-C₆ cycloalkyl, C₁-C₆ haloalkyl, C₂-C₆ alkenyl, C₂-C₆ alkynyl, heterocyclyl having 5 to 10 ring atoms, C₅-C₁₀ aryl, heteroaryl having 5 to 10 ring atoms, C₇-C₁₆ aralkyl, and —NR″R″′; said alkyl, cycloalkyl, hetroalkyl, haloalkyl, alkenyl, alkynyl, heterocyclyl, aryl, heteroaryl and aralkyl being optionally substituted with one or more substituents independently selected from oxo, halogen, C₁-C₆ alkyl, C₁-C₆ heteroalkyl, C₃-C₆ cycloalkyl, C₁-C₆ haloalkyl, heterocyclyl having 5 to 10 ring atoms, C₅-C₁₀ aryl, heteroaryl having 5 to 10 ring atoms, —OH, —NR″R′″, —NO₂, —CN, —O—(CO)—R and —(CO)—R;

with each R being independently selected from H, C₁-C₆ alkyl, C₁-C₆ alkoxy and —NR″R″′; and

with each R″ and R″′ being independently selected from H and C₁-C₆ alkyl.

Preferably, R₁₁ is selected from C₁-C₆ alkyl, NR″R″′, and C₁-C₆ heteroalkyl, said alkyl, and heteroalkyl being optionally substituted with one or more substituents independently selected from oxo, heteroaryl having 5 to 10 ring atoms, C₅-C₁₀ aryl, C₁-C₆ heteroalkyl, —O—(CO)—R and —OH; with R being selected from H and C₁-C₆ alkyl and with R″ and R″′ being independently selected from H and C₁-C₆ alkyl, more preferably R₁₁ is selected from

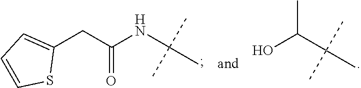

The cation Q can be selected from Na⁺, K⁺, Li⁺, and NH4⁺.

According to a specific embodiment, Z is S and n is 1. According to another specific embodiment, Z is —CR₉R₁₀— and n is 0; with R₉ and R₁₀ being identical or different and representing, independently of each other, a hydrogen atom or a C₁-C₄ alkyl.

According to a specific embodiment, compound (I) is of formula (Ic):

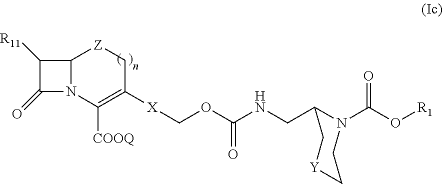

where R₁, R₁₁, Z, Q, n, X and Y and n are as defined for compound of formula (I).

According to a specific embodiment, compound (I) is of formula (Id):

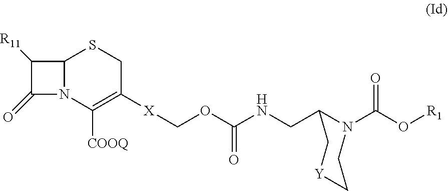

where R₁, R₁₁, Q, X, and Y are as defined for compound of formula (I).

Compound of formula (Id) can be of formula (Id′), (Id″) or (Id″′):

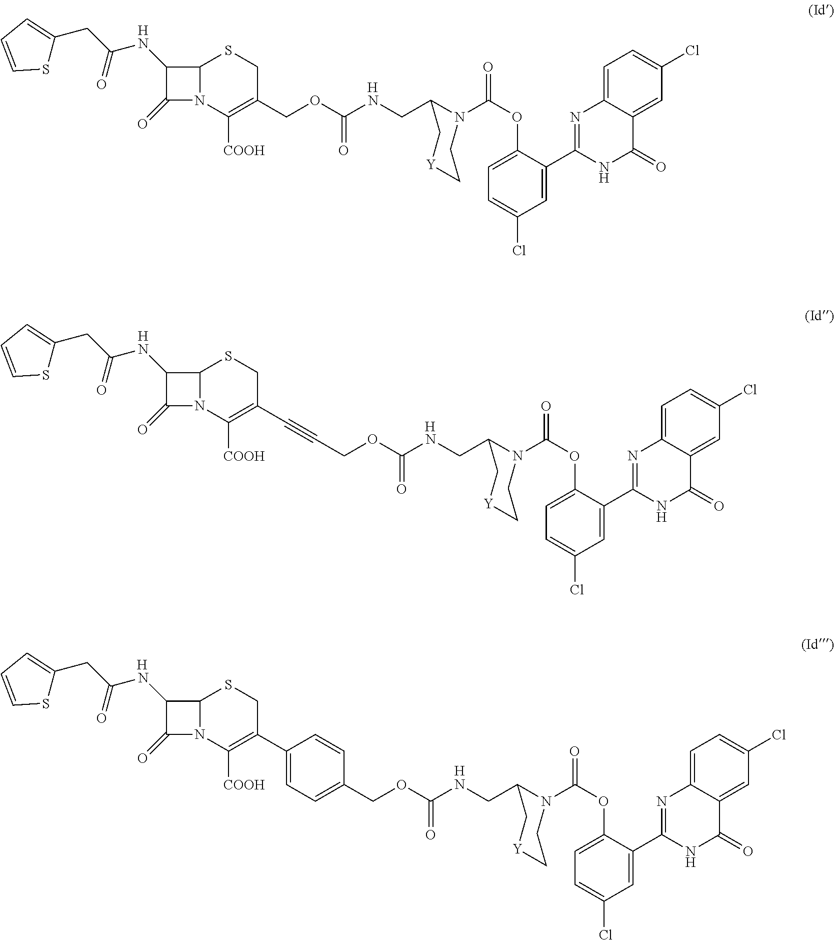

where Y is as defined for compound of formula (I)

Compound of formula (Id) can be selected from the following compounds:

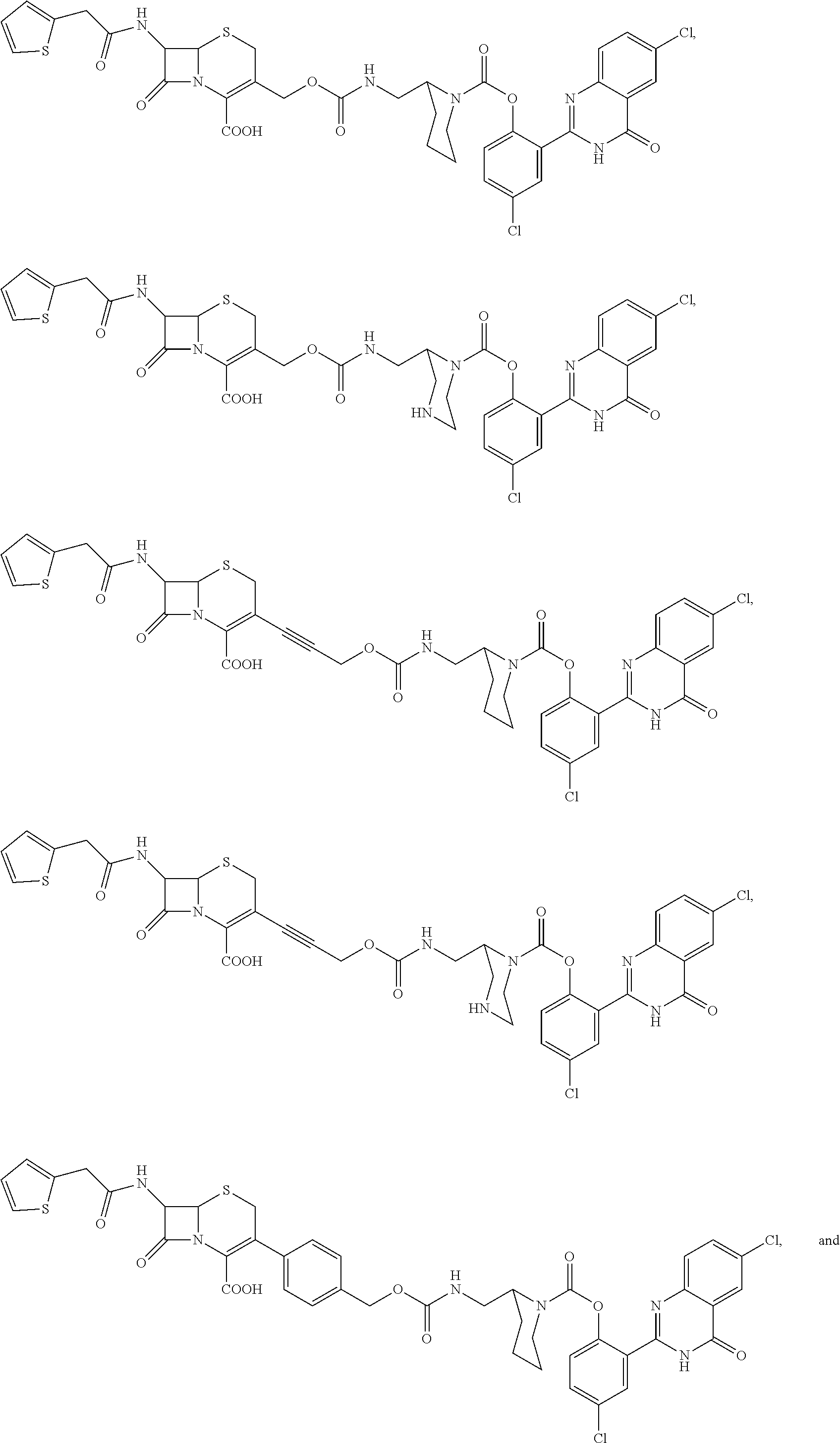

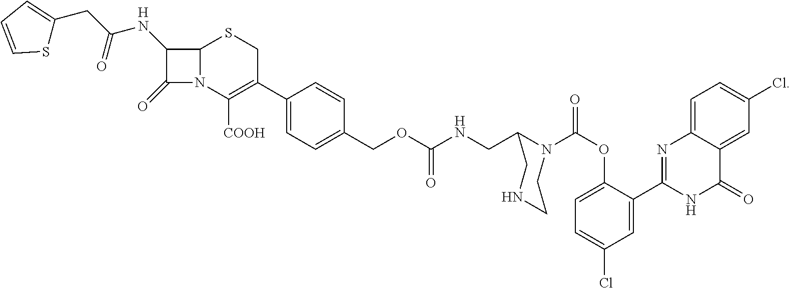

According to a specific embodiment, compound (I) is of formula (le):

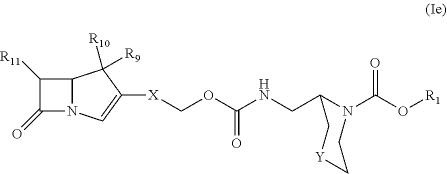

where R₁, R₉, R₁₀, R₁₁, Q, X and Y are as defined for compound of formula (I).

Compound of formula (Ie) can be of formula (Ie′), (Ie″) or (Ie″ ):

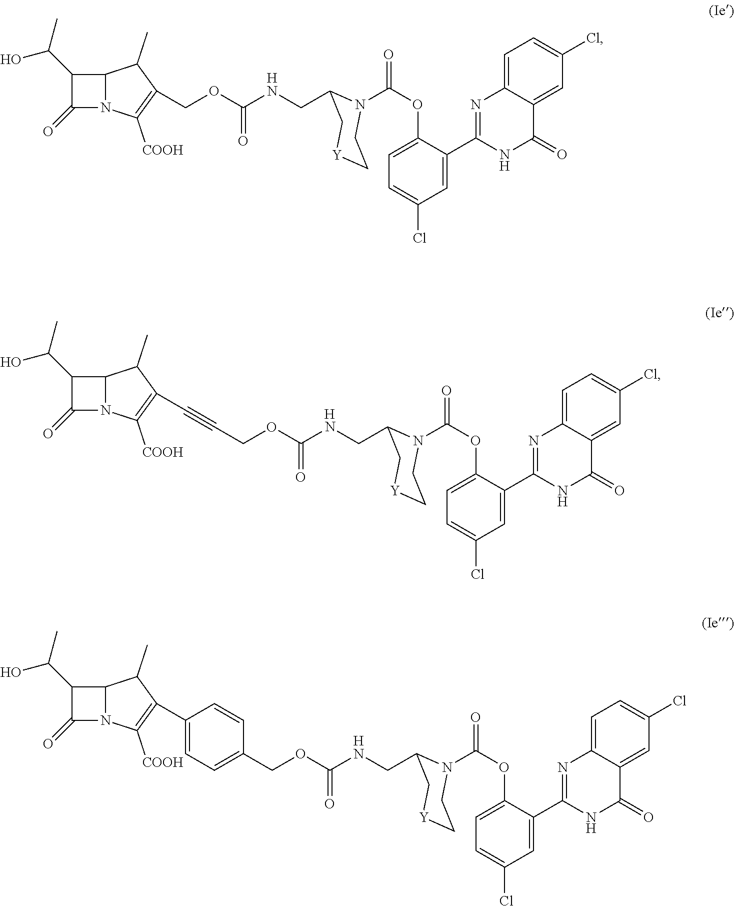

where Y is as defined for compound of formula (I).

Compound of formula (Ie) can be selected from the following compounds:

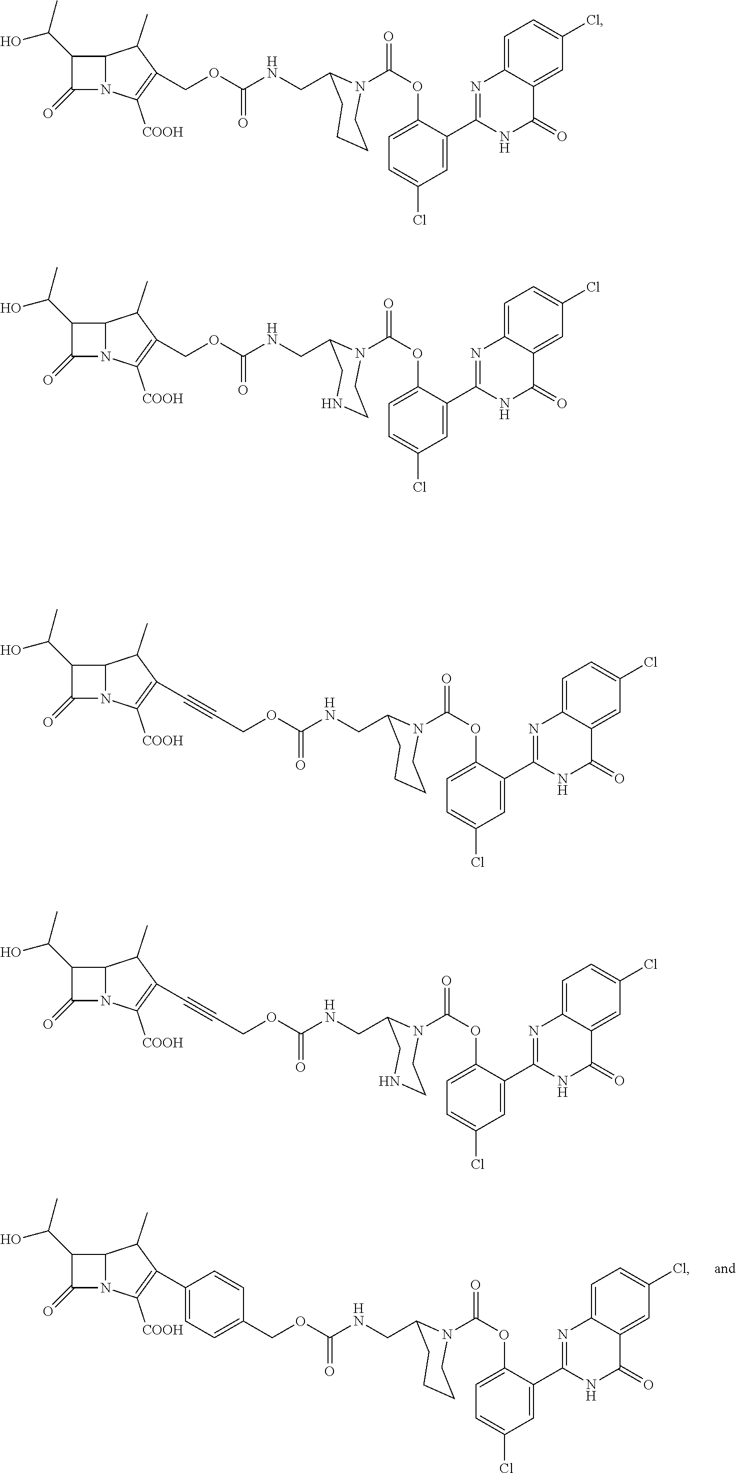

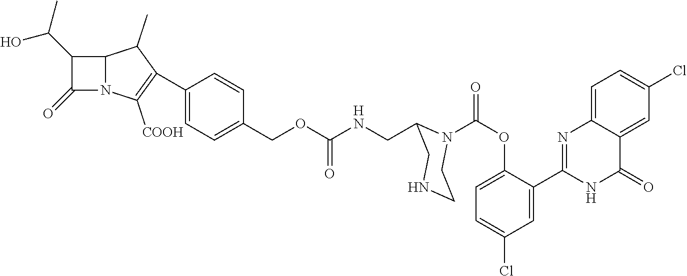

According to an embodiment, in the compound of formula (I):
W is —O—;
R₁ is such that HOR₁, obtained after cleavage of the —C(O)—OR₁ bond present in formula (I), belongs to the class of fluorophores, preferably to the class of fluorophores leading to an intramolecular proton transfer in an excited state, and —OR₁ corresponds to a group of formula (A1):

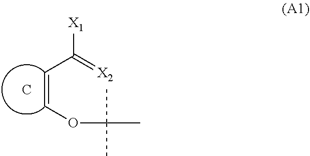

in which:
either X₂ is an oxygen atom and X₁ is a —NH₂, —OH, —SH, C₁-C₂₀ alkyl, C₅-C₂₄ aryl, C₂-C₆ alkenyl, —O—(C₁-C₂₀ alkyl), —O-phenyl, —NH-(C₁-C₂₀ alkyl), —NH-phenyl, —S-(C₁-C₂₀ alkyl) or —S-(C₅-C₂₄ aryl group), said alkyl, aryl, alkenyl and phenyl groups being optionally substituted;

or X₂ represents a nitrogen atom and is bound to X₁ which then represents CH, O, S, N or, NH to form a C₅-C₂₄ heteroaryl, optionally substituted;

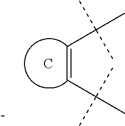

represents a C₅-C₂₄ aryl or a C₅-C₂₄ heteroaryl, optionally substituted, for example, chosen from among the phenyl, naphtyl groups, and:

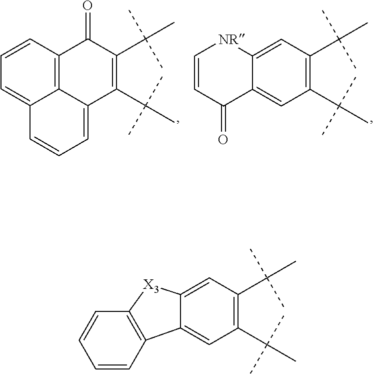

said groups being optionally substituted;

with X₃ which represents S, O or NRd and Rd which represents a hydrogen atom or a C₁-C₄ alkyl group;
R₂, R₃ and R₄ are defined as follows:
   R₃ is a C₁-C₄ alkyl or a hydrogen atom and R₂ and R₄ are bonded to each other and form a —(CH₂)ₚ—Y_{q}—(CH₂)ᵣ— chain in direction of R₂ toward R₄, wherein
      Y is O, NR₁₄, N(R₁₄)₂⁺or S, preferably NR₁₄ or N(R₁₄)₂⁺,
      p=0, 1, 2, 3, 4 or 5,
      q=0 or 1,
      r=0, 1, 2, 3, 4 or 5,
      p+q+r=4, 5, or 6, and
      each R₁₄ represents independently a hydrogen atom, a C₁-C₆ alkyl, an amino protecting group or -(L)_{q}-GP, with q which is equal to 0 or 1, L which is a linking arm and GP which is a hydro solubilizing group;
R₅ and R₆ are identical or different and represent, independently of each other, a hydrogen atom, a C₁-C₄ alkyl, or a C₅-C₁₀ aryl;
R₇ is a hydrogen atom, or a group selected from C₁-C₄ alkyl and C₁-C₄ alkoxy;
R₈ represents a hydrogen atom;
V represents an oxygen atom;
n is 0 or 1;
Z is —S— or —CR₉R₁₀—; with R₉ and R₁₀ being identical or different and representing, independently of each other, a hydrogen atom or a C₁-C₄ alkyl;
Q is H or a cation; and
R₁₁ is selected from C₁-C₆ alkyl, C₁-C₆ heteroalkyl, C₃-C₆ cycloalkyl, C₁-C₆ haloalkyl, C₂-C₆ alkenyl, C₂-C₆ alkynyl, heterocyclyl having 5 to 10 ring atoms, C₅-C₁₀ aryl, and heteroaryl having 5 to 10 ring atoms, C₇-C₁₆ aralkyl;

said alkyl, cycloalkyl, hetroalkyl, haloalkyl, alkenyl, alkynyl, heterocyclyl, aryl, heteroaryl and aralkyl being optionally substituted with one or more substituents independently selected from oxo, halogen, C₁-C₆ alkyl, C₁-C₆ heteroalkyl, C₃-C₆ cycloalkyl, C₁-C₆ haloalkyl, heterocyclyl having 5 to 10 ring atoms, C₅-C₁₀ aryl, heteroaryl having 5 to 10 ring atoms, —OH, —NR″R″′, —NO₂, —CN and —(CO)—R; with each R being independently selected from H, C₁-C₆ alkyl, C₁-C₆ alkoxy and —NR″R″′; and

with each R″ and R″′ being independently selected from H and C₁-C₆ alkyl,
X represents a bond, or a group selected from

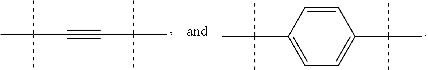

### Compound of Formula (II)

The invention also relates to a compound of formula (II):

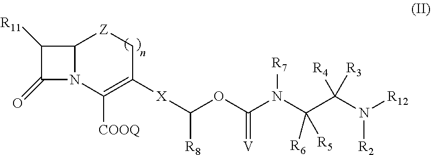

in which:
R₂, R₃, R₄, R₅, R₆, R₇, R₈, R₁₁, Z, n, Q, X and V are as defined for compound of formula (I),
R₁₂ represents a hydrogen atom, or an amine function protecting group.

The compounds of formula (II) are synthesis intermediates of the compounds of formula (I), by amine functions protective group is understood protective groups such as those described in Protective Groups in Organic Synthesis, Greene T. W. et Wuts P. G. N., ed. John Wiley and Sons, 2006 and in Protective Groups, Kocienski P. J., 1994, Georg Thieme Verlag.

According to an embodiment, R₁₂ is an amine functions protective group. As an example, R₁₂ represents an amine function protective group chosen from among the allyl or carbamate groups, such as a tert-butoxycarbonyl (Boc) group, fluorophenyl methoxycarbonyl (Fmoc) group, allyloxy carbonyl (Alloc) group or 2,2,2-trichloroethoxycarbonyl (Troc) group.

According to a specific embodiment, R₁₂ represents a hydrogen atom.

In a specific embodiment, compound of formula (II) is of formula (IIa):

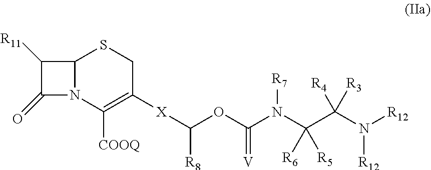

where R₃, R₄, R₅, R₆, R₇, R₈, R₁₁, Q, X, and Y are as defined for compound of formula (I) and R₁₂ is as defined for compound of formula (II).

In another specific embodiment, compound of formula (II) is of formula (IIb):

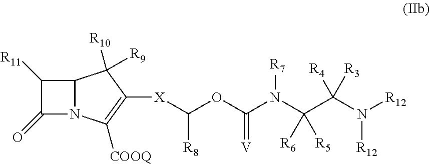

where R₃, R₄, R₅, R₆, R₇, R₈, R₉, R₁₀, R₁₁, Q, X and Y are as defined for compound of formula (I) and R₁₂ is as defined for compound of formula (II).

Advantageously, the compound of formula (II) is of formula (IIc):

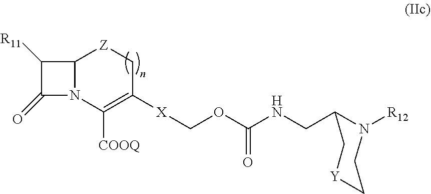

where R₁₁, Z, Q, n, X and Y are as defined for compound of formula (I) and R₁₂ is as defined for compound of formula (II).

In a specific embodiment, compound of formula (II) is of formula (IId):

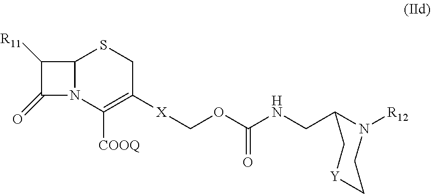

where R₁₁, Q, X and Y are as defined for compound of formula (I) and R₁₂ is as defined for compound of formula (II).

In another specific embodiment, compound of formula (II) is of formula (IIe):

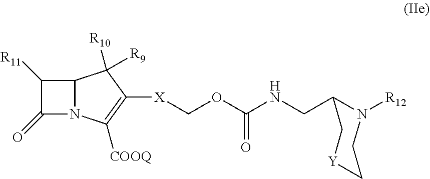

where R₁₁, Q, X and Y are as defined for compound of formula (I) and R₁₂ is as defined for compound of formula (II).

### Process for the Preparation of the Compounds of Formula (I)

This invention also concerns a process for the preparation of a compound of formula (I) comprising the following steps:
implementation of a compound (II),
implementation of a compound (III) of formula

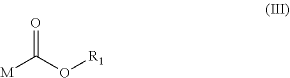

with R₁ as defined for compound (I) and M representing a leaving group, preferably selected from a halogen atom, and in particular Cl, an imidazolyl group, a triazolyl group, and a para-nitrophenoxy, and more preferably with M representing a para-nitrophenoxyl.
obtaining compound (I) by addition reaction of said compound (II) to compound (III)

This process is particularly suitable when X is a bond.

According to one embodiment, the reaction of addition of compound (II) to compound (III) is executed with a compound (II) in which R₁₂ is a hydrogen atom.

According to another embodiment, we have available a compound (II) in which R₁₂ is not a hydrogen atom, and a step of deprotecting the amine function of the compound (II) is executed prior to the reaction of addition of compound (II) to compound (III) so as to obtain a compound (II) such as R₁₂=H.

When V═O, compound (II) can be beneficially obtained according to the following steps:
implementation of a compound (V) of the following formula:

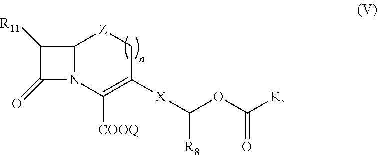

implementation of a compound (VI) of formula

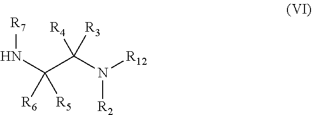

and obtaining compound (II) by addition reaction of said compound (VI) to the compound (V),

where R₈, R₁₁, Q, X and Z are as defined in the context of the invention, and K represents a leaving group, in particular a halogen, and specifically chlorine, or an imidazolyl or para-nitrophenyl group.

According to a specific embodiment, the compound of formula (V) is of formula (Va):

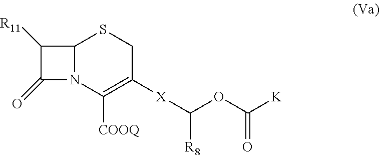

where R₈, R₁₁, Q, Z, X and K are as defined in the context of the invention. According to another specific embodiment, the compound of formula (V) is of formula (Vb):

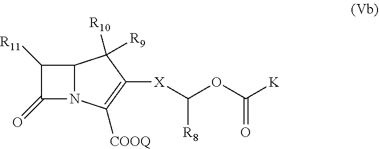

where R₈, R₉, R₁₀, R₁₁, Q, X and K are as defined in the context of the invention.

This invention also concerns a process for the preparation of a compound of formula (I) when X is

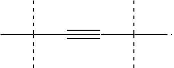

In this case, the compound can be prepared using a Sonogashira coupling reaction, a well known reaction for the person skilled in the art. This process can comprise the following steps:
implementation of a compound (VII) of the following formula:

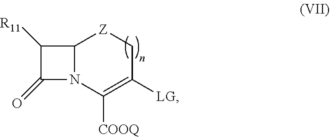

implementation of a compound (VIII) of formula

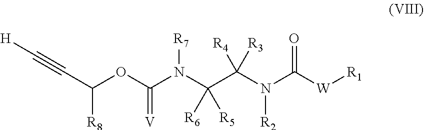

and obtaining compound (I) by reaction of said compound (VII) with compound (VIII),

Where R₁, R₂, R₃, R₄, R₅, R₆, R₇, R₈, V, R₁₁, Q, W, Z and n are as defined in the context of the invention, and LG represents a leaving group, preferably selected from halogen and trifluoromethanesulfonate (triflate).

This process can be implemented using standard conditions like a palladium catalyst and a copper co-catalyst.

This invention also concerns a process for the preparation of a compound of formula (I) when X is

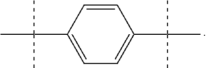

In this case, the compound can be prepared using a Suzuki coupling reaction, a well known reaction for a person skilled in the art. This process can comprise the following steps:
implementation of a compound (VII) of the following formula:

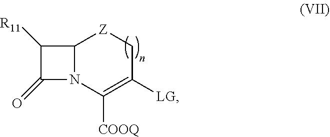

implementation of a compound (IX) of formula

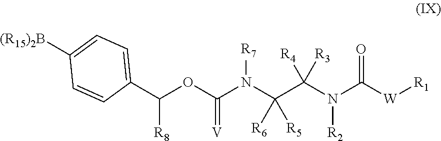

and obtaining compound (I) by reaction of said compound (VII) with compound (IX),

Where R₁, R₂, R₃, R₄, R₅, R₆, R₇, R₈, V, R₁₁, Q, W, Z and n are as defined in the context of the invention, LG represents a leaving group, preferably selected from halogen and trifluoromethanesulfonate (triflate), and each R₁₅ represents OH, or both R15 are bonded to each other and forms, together with the B atom to which they are bonded, a heterocycle having from 5 to 10 ring atoms. Examples of R15 groups that are bonded to each other include pinacol, catechol, and methyliminodiacetate.

This process can be implemented using standard conditions, like a Palladium catalyst and a base.

This invention also concerns a process for the preparation of a compound of formula (I) when X is

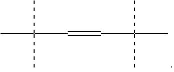

In this case, the compound can be prepared using a Suzuki coupling reaction, a well known reaction for a person skilled in the art. This process can comprise the following steps:
implementation of a compound (VII) of the following formula

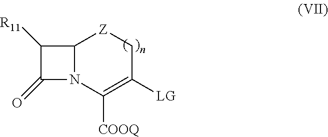

implementation of a compound (X) of formula

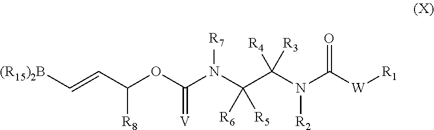

and obtaining compound (I) by reaction of said compound (VII) with compound (X),

Where R₁, R₂, R₃, R₄, R₅, R₆, R₇, R₈, V, R₁₁, Q, W, Z and n are as defined in the context of the invention, LG represents a leaving group, preferably selected from halogen and trifluoromethanesulfonate (triflate), and R₁₅ represents OH, or both R15 are bonded to each other and forms, together with the B atom to which they are bonded, a heterocycle having from 5 to 10 ring atoms. Examples of R15 groups that are bonded to each other include pinacol, catechol, and methyliminodiacetate.

This process can be implemented using standard conditions, like a Palladium catalyst and a base.

The compound of formula (X) can be obtained from the compound of formula (VIII) by hydroboration of the triple bond. The double bond in the compound of formula (X) can be E or Z.

### Detection of the Presence of β-Lactamase

The invention also concerns a method for the in vitro or ex vivo detection of a β-lactamase, comprising the steps of:
putting a sample to be analyzed into contact with a compound (I),
applying suitable conditions in order to make possible the formation of a fluorescent precipitate by cleavage of the covalent bond between C(═V) and NR₇, followed by a cleavage of the —C(O)—WR₁, bond, leading to the release of HWR₁,
quantitative or qualitative analysis of the fluorescent precipitate, and
correlating the quantitative or qualitative analysis of the fluorescent precipitate to the presence or absence of β-lactamase in the sample.

The invention also concerns a method for the in vitro or ex vivo detection of antibiotic-resistant bacteria comprising the steps of:
putting a sample to be analyzed into contact with a compound (I),
applying suitable conditions in order to make possible the formation of a fluorescent precipitate by cleavage of the covalent bond between C(═V) and NR₇, followed by a cleavage of the —C(O)—WR₁, bond, leading to the release of HWR₁,
quantitative or qualitative analysis of the fluorescent precipitate, and
correlating the quantitative or qualitative analysis of the fluorescent precipitate to the presence or absence of antibiotic-resistant bacteria in the sample.

The invention also concerns a kit for detecting a β-lactamase, said kit comprising a compound (I).

The invention also concerns a device for detecting a β-lactamase said device comprising a compound (I). Preferably, the device is an in-vitro diagnostic medical device (IVD).

According to an embodiment, the β-lactamase is a carbapenemase.

The invention also concerns a method for the in vitro or ex vivo detection of a carbapenemase, comprising the steps of:
putting a sample to be analyzed into contact with a compound (Ib),
applying suitable conditions in order to make possible the formation of a fluorescent precipitate by cleavage of the covalent bond between C(═V) and NR₇, followed by a cleavage of the —C(O)—WR₁, bond, leading to the release of HWR₁,
quantitative or qualitative analysis of the fluorescent precipitate, and
correlating the quantitative or qualitative analysis of the fluorescent precipitate to the presence or absence of carbapenemase in the sample.

The compounds of formula (I) according to the invention may also be used to detect a β-lactamase, in vivo, in animals or in human beings.

The administration of the compound of formula (I) can be completed by an intravenous or intra-peritoneal injection, or cutaneously, by use of a spray containing the molecule in solution, for example.

Analysis of the fluorescence of the compound of formula (I) may take place in an imaging chamber using fluorescence or epi-fluorescence type tomography techniques.

The invention also concerns a method for detecting, in vitro or ex vivo, the presence of a β-lactamase by means of the compound (I) according to the invention.

The sample can be any suitable biological sample, from a human being, an animal, a plant or a micro-organism. In the case of a sample from a human being or an animal, this may specifically be a sample of a biological fluid, specifically a sample of whole blood, serum, plasma, urine, a tissue sample, or a sample of isolated cells, and in particular, of a cellular medium. In the case of a sample from a plant, this can be a plant extract, an extract of a fungus or of algae, of living cells, and in particular, of a cellular medium. It is also possible for the sample to directly comprise the plant. In the case of a sample from a micro-organism, the micro-organism can be a bacterium, a virus, a fungus or a yeast, and can also be a micro-biota. The sample may directly comprise the micro-organism, or and extract of the latter, or even the culture medium in which the micro-organism was incubated. In all cases, the sample can be used as is, or can be submitted, before being put in the presence of the probe, to an enriching or culturing type preparation, well known to the person skilled in the art.

Analysis of the compound or fluorescent precipitate can comprise:
a step of exposing the fluorescent precipitate to a light source capable of producing light at an absorption wavelength of the fluorescent precipitate, and
a step of detecting the fluorescence of the resulting precipitate.

The analysis may also comprise a step, subsequent to the step of detection of the fluorescence, of sorting analyzed samples based on the signal provided by said fluorescent precipitate. The sorted samples can be colonies of micro-organisms, separated in space, such as dishes of micro-biological cultures. The sorted samples can also be small objects, liquids, solids, gelatinous or of heterogeneous composition, containing either bio-molecules or colonies of micro-organisms. When detection is done in parallel on several samples, the sorting can be done, for example, by diversion of a flow of samples set into motion in a device making it possible to sort according to an optical signal, representative of the emitted fluorescence, such as flow cytometry or a digital milli- or micro-fluid device.

This invention makes the activity of β-lactamases accessible by fluorescent imaging using fluorophores, preferably ESIPT fluorophores. Beneficially, no background noise due to spontaneous degradation (that is, in the absence of the target β-lactamase, in a physiological medium) was observed. The probe itself is slightly fluorescent, or not at all fluorescent, in particular at the wavelength of emission of the fluorophore fiber on which the detection/imaging instrument is set. Thus, the probe functions in an on/off mode and can be used for the development of analyses with maximum sensitivity.

Probes according to the invention are interesting for several high sensitivity applications in the life sciences, specifically: (1) high yield targeting of β-lactamase activity expressed by bacterial colonies on an agar plate (analysis of colonies); (2) the in vitro detection of β-lactamase in biological liquids (hematology and others); (3) visualization of a β-lactamase activity at the level of a simple cell in flow cytometry; (4) the detection of sub-cellular β-lactamase in cultivated cells (confocal fluorescence microscopy); (5) the histo-chemical detection of β-lactamase (at the tissue level); and finally (6), in vivo imagery of an entire animal.

Thus, the compounds of formula (I), as β-lactamase substrates according to this invention, have a large number of potential applications. Examples of these applications include the design of analyses of bacterial colonies. These are currently executed on an agar dish (Petri dish) where up to 3,000 colonies can be distinguished without having to actively separate them into separate compartments such as the wells contained in a multi-well dish. Thus, it is possible to (1) design tests on clinical samples making it possible to identify from among a group of bacterial lines a pathogenic line of interest and (2) to complete large-scale parallel tests of a bank of self-produced proteins expressed by a classic bacterial host (often commercial). This collection of proteins can be understood to contain a protein of specific interest, for example, a β-lactamase with a selectivity for a specific β-lactam group, or a β-lactamase hydrolyzing a β-lactam. In the field of directed evolution of β-lactamase or enzymes in particular, there is high demand for effective and sensitive analyses for sieving very large numbers of protein variants, easily exceeding 10⁶. The application of the probe according to the invention can be most easily envisaged by dissolution in the agar solution before it is poured into the dish or gelifies itself. As an alternative, substrates are incubated with colonies by immersion of a filter before they are introduced into colonies. The principal benefit which the probe according to the invention contributes to such an analysis of colonies is the on-site precipitation of the fluorophore; dilution of the fluorescent signal is therefore very reduced, which makes long incubation periods possible and therefore, greater sensitivity for analysis. The very large Stokes shift of dichloro-HPQ (approximately 140 nm), or of any analog of HPQ, should not be mis-estimated; it also contributes to superior sensitivity, and the emitted fluorescence is easily distinguishable from the native fluorescence which could come from the biological sample.

Probes according to the invention can also be used for macroscopic fluorescence imaging, namely, for the entire organism. In this case, the probe will penetrate the cell wall in order to reach the activity of interest.

Examples, in relation to the annexed figures, make it possible to illustrate the invention, but not in a limitative way.

### EXAMPLES

### Example 1: Synthesis of Compound 15

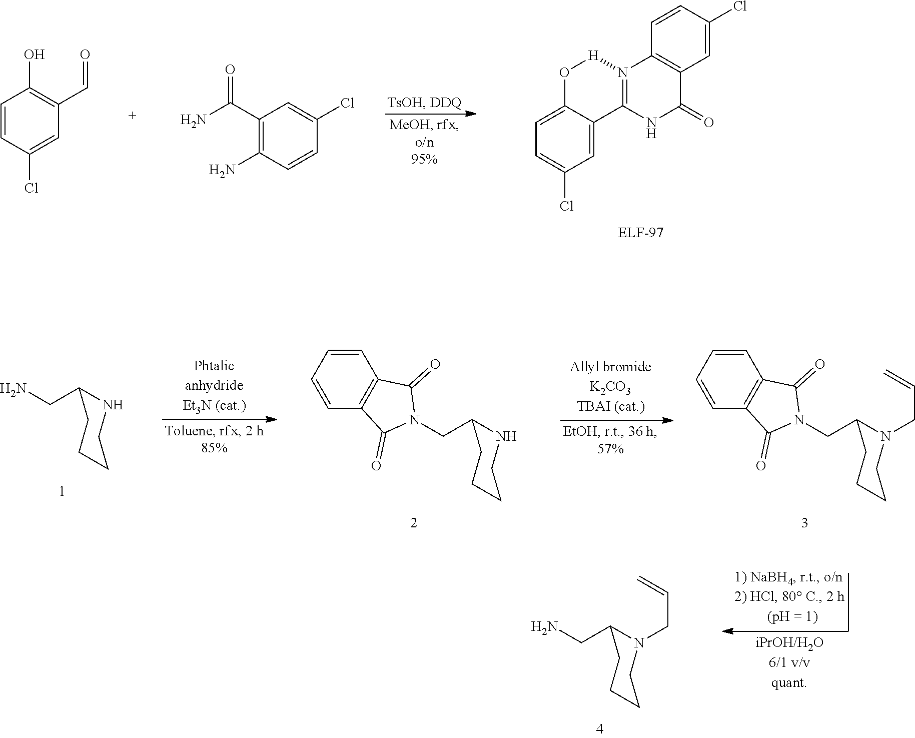

### ELF-97

A solution of anthranilamide (2.000 g, 11.7 mmol, 1.0 eq.) in dry EtOH (20 mL) is treated with 5-chlorosalicylaldehyde (1.831 g, 11.7 mmol, 1.0 eq.), and the mixture is refluxed for 30 minutes. Then, para-toluenesulfonic acid (PTSA) (40 mg, 0.234 mmol, 0.02 eq.) is added, and refluxing is continued for another hour. The reaction mixture is brought down to room temperature and treated portionwise with 2,3-dichloro-5,6-dicyano-1,4-benzoquinone (DDQ) (2.678 g, 11.8 mmol, 1.01 eq.). Stirring is continued overnight. The resulting crude suspension is filtered and the filter cake washed 2 times with EtOH and 2 times with diethyl ether. ELF-97 (3.41 g, 11.11 mmol, 95%) is obtained as a light beige powder and used without further purification in a later step.

¹H-NMR (300 MHz, CDCl₃): δ (ppm)=13.38 (s, 1H), 12.64 (s, 1H), 8.29 (s, 1H), 8.10 (s,1H), 7.88 (q, J=7.8 Hz, 2H), 7.49 (d, J=7.6 Hz,1H), 7.05 (d, J=8.8 Hz, 1H)

Spectral data are in accordance with literature values (M. Prost, L. Canaple, J. Samarut, J. Hasserodt. Tagging Live Cells that Express Specific Peptidase Activity with Solid-State Fluorescence. ChemBioChem 2014, 15, 1413-1417).

### Compound 4

A solution of 2-aminomethylpiperidine (1) (3.0 g, 26.3 mmol, 1.0 eq.) in toluene (50 mL) is treated portionwise with phthalic anhydride (3.89 g, 26.3 mmol, 1.0 eq.), followed by dropwise addition of triethylamine (550 μL, 3.95 mmol, 0.15 eq.). The mixture is refluxed for 2 h using a Dean-Stark apparatus. The mixture is filtered and the filtrate reduced to dryness under reduced pressure. The product 2 (5.42 g, 22.2 mmol, 85%) is obtained as a light yellow solid and used in the next step without further purification.

An ice-cold ethanol solution (45 mL) of compound 2 (5.42 g, 22.2 mmol, 1.0 eq.) is treated with potassium carbonate (3.99 g, 28.9 mmol, 1.3 eq.), tetra-n-butylammonium iodide (820 mg, 2.2 mmol, 0.10 eq.), and allyl bromide (2.50 mmL, 28.9 mmol, 1.3 eq.). The cooling bath is removed and the mixture stirred for 36 h. Upon verification that the reaction is complete, the mixture is filtered over a pad of Celite and the filtrate evaporated to dryness under reduced pressure. The oily residue is taken up in EtOAc and washed with a saturated aqueous solution of NH₄Cl; the two layers are separated, and the organic phase is washed twice with saturated aqueous NH₄Cl. The combined aqueous phases are extracted 3 times with EtOAc. The combined organic phases are dried over Na₂SO₄, filtered and evaporated to dryness. The crude oil is purified via column chromatography on silica gel (PE/EtOAc 80/20 to 60/40 v/v) to yield 3 (3.582 g, 12.6 mmol, 57%) as a light yellow oil.

¹H -NMR (300 MHz, CDCl₃): δ (ppm)=7.89-7.82 (m, 2H), 7.75-7.68 (m, 2H), 3.68 (d, J=4 Hz, 2H), 3.13-3.05 (m, 1H), 2.98-2.88 (m, 1H), 2.64-2.54 (m, 1H), 1.87-1.78 (m, 1H), 1.76-1.68 (m, 1H), 1.63-1.54 (m, 1H), 1.45-1.34 (m, 2H), 1.30-1.15 (m, 1H).

¹³C-NMR (75 MHz, CDCl₃): δ(ppm)=168.4, 133.7, 131.9, 123.0, 55.5, 46.4, 43.4, 30.6, 26.1, 24.1.

HRMS: ESI: [M+H]⁺ m/z found 245.1290, calc. 245.1290.

An ice-cold solution of 3 (3.582 g, 12.6 mmol, 1.0 eq.) in iPrOH/H₂O 6/1 v/v (175 mL) is treated portionwise with sodium borohydride (665 mg, 17.58 mmol, 5.0 eq.). Cooling is removed, and the Mixture stirred overnight (o/n) at room temperature. The pH is then adjusted to 1 using concentrated HCl. The resulting mixture is filtered and the filtrate heated to 80° C. for 2 h. The isopropyl alcohol is removed under reduced pressure and the resulting aqueous solution washed 5 times with diethyl ether, basified With 2 M NaOH aqueous solution, and extracted With diethyl ether. The combined organic extracts are dried over Na₂SO₄, filtered and evaporated to dryness to obtain 4 as a light yellow oil (1.939 g, 12.6 mmol, quantitative yield).

¹H -NMR (300 MHz, CDCl₃): δ (ppm)=5.99-5.86 (m, 1H), 5.24-5.13 (m, 2H),3.41(ddt, J=14 Hz, J=6Hz, J=1.5 Hz, 11H), 3.04-2.90 (m,3H), 2.74 (dd, J=13 Hz, J=3 Hz, 1H), 2.21 (tt, J=9.6 Hz, J=3.3H, 2 Hz), 1.80-1.71 (m, 1H), 1.68-1.43 (m, 2H), 1.39-1.25 (m, 3H).

¹³C -NMR (75 MHz, CDCl₃): δ(ppm)=157.34, 134.50, 117.71, 58.62, 56.29, 51.90, 42.31, 28.85, 24.92, 23.58.

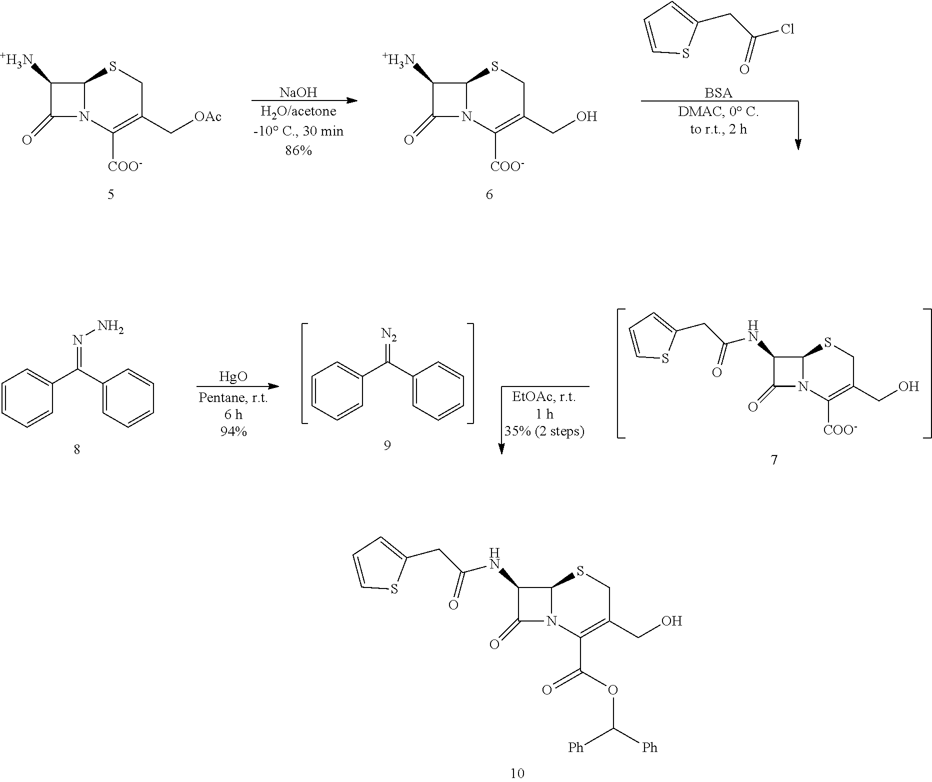

HRMS: ESI: [M+H]⁺ m/z found 155.1543, calc. 155.1548.

### Compound 6

A solution of 7-aminocephalosporanic acid (5) (2.000 g, 7.345 mmol, 1.0 eq.) in H₂O/MeOH (20 mL, 1/1 v/v) at −20° C. is treated with 10 M NaOH (2 mL) and the resulting mixture stirred at −20° C. for 30 min. The pH is adjusted to 3 with concentrated HCl. The temperature is now brought to 0° C. and the light yellow precipitate thus formed is filtered off, washed with MeOH, acetone, and ether, and then dried. The desired alcohol 6 is obtained as an off-white powder (1.451 mg, 6.302 mmol, 86%).

¹H -NMR (300 MHz, DMSO): δ (ppm)=4.83 (AB system, Δμ=59 Hz, J=5 Hz, 2H), 4.21 (AB system, Δμ=17 Hz, J=13 Hz, 2H), 3.52 (AB system, Δμ=26 Hz, J=18 Hz, 2H).

Spectral data are in accordance with literature values (S. Desgranges, C. C. Ruddle, L. P. Burke, T. M. McFadden, J. E. O'Brien, D. Fitzgerald-Hughes, H. Humphreys, T. P. Smyth, M. Devocelle. β-Lactam-host defense peptide conjugates as antibiotic prodrug candidates targeting resistant bacteria. RSC Advances 2012, 2, 2480).

A solution of benzophenone hydrazone (8) (4.906 g, 25.00 mmol, 1.0 eq.) in PE (30 mL) is treated with mercury oxide (II) (25.469 g, 25.25 mmol, 1.01 eq.) and the resulting mixture stirred for 6 h at room temperature and under daylight exclusion. The resulting purple mixture is filtered to remove mercury-containing residues and the resulting solution evaporated under reduced pressure. The purple liquid containing the target reagent diazodiphenylmethane 9 (4.570 g, 23.53 mmol, 94%) is taken up in EtOAc (15 mL) and quickly used in the next step without further purification.

A solution of compound 6 (5.000 g, 21.72 mmol, 1.0 eq.) in dimethylacetamide (DMAC) (80 mL) is treated with bis(trimethylsilyl)acetamide (BSA) (13.3 mL, 54.29 mmol, 2.5 eq.) and the resulting mixture stirred for 30 min at room temperature. The clear solution is cooled to −30° C. and 2-thiopheneacetyl chloride (3.48 mL, 28.23 mmol, 1.3 eq.) is added dropwise. The resulting mixture is stirred for 2 h at −20° C., poured onto ice water and extracted with EtOAc. The combined organic phases are washed with brine, dried over Na₂SO₄ and their volume adjusted to 80 mL under reduced pressure. The solution is cooled to 0 ° C. and treated with the above diazodiphenylmethane 9 solution in EtOAc (4.429 g, 22.80 mmol, 1.05 eq.) until the purple color persists. The volume of the resulting solution is reduced under vacuum before being added dropwise to a solution of pentane (300 mL), thus causing the precipitation of a light yellow solid. The latter is filtered off to give the doubly protected product 10 (3.957 g, 7.601 mmol, 35% over two steps).

¹H -NMR (300 MHz, CDCl₃): δ (ppm)=9.23-9.12 (m, 1H), 7.54-7.46 (m, 11H), 7.01-6.88 (m, 3H), 5.89-5.67 (m, 1H), 5.20-5.02 (m, 2H), 4.21 (d, J=4 Hz, 1H), 3.78 (s, 2H), 3.62 (s, 1H), 2.95 (s, 1H), 2.79 (s, 1H).

Spectral data are in accordance with literature values (S. Desgranges, C. C. Ruddle, L. P. Burke, T. M. McFadden, J. E. O′Brien, D. Fitzgerald-Hughes, H. umphreys, T. P. Smyth, M. Devocelle. +-Lactam-host defence peptide conjugates as antibiotic Prodrug candidates targeting resistant bacteria. RSC Advances 2012, 2, 2480).

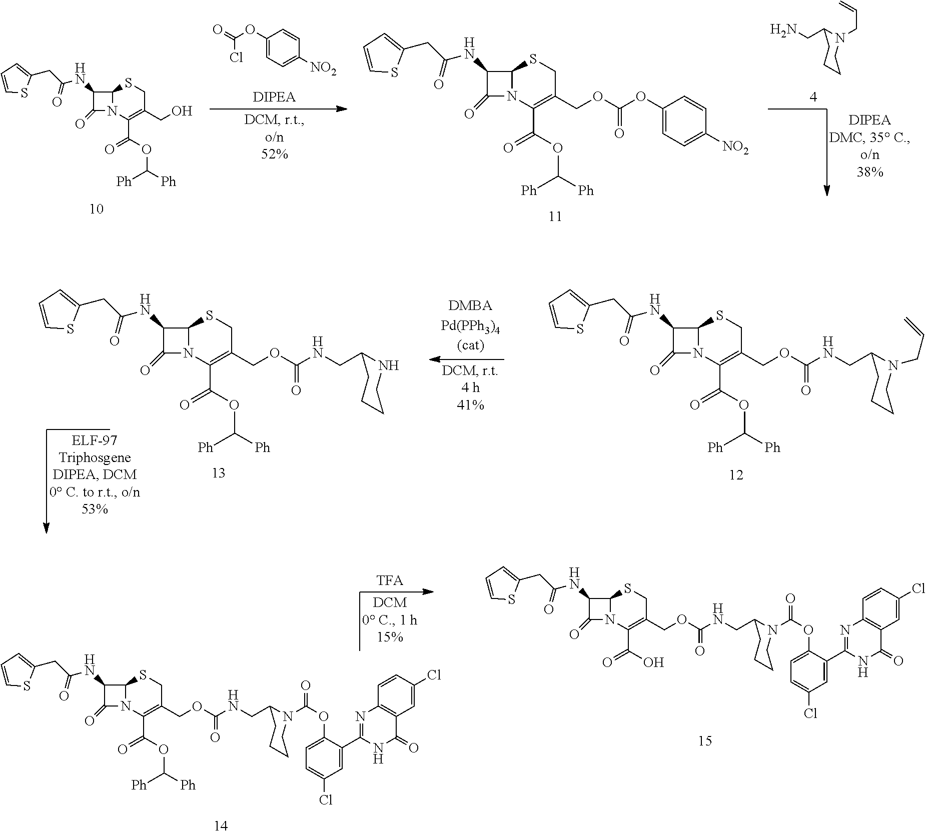

### Compound 15

An ice-cold solution of alcohol 10 (1.000 g, 1.921 mmol, 1.0 eq.) in DCM (50 mL) is treated with 4-nitrophenyl chloroformate (775 mg, 3.842 mmol, 2.0 eq.), pyridine (155 μL, 1.921 mmol, 1.0 eq.) and 4-dimethylaminopyridine (DMAP) (24 mg, 0.192 mmol, 0.1 eq.). The resulting mixture is stirred for 2 h at room temperature, then washed with water, dried over Na₂SO₄ and concentrated under reduced pressure. The crude residue is purified by flash column chromatography on silica gel (PE/EtOAc 70/30) to furnish the desired carbonate 11 as a light yellow solid (685 mg, 0.999 mmol, 52%).

¹H -NMR (300 MHz, CDCl₃): δ (ppm)=8.26 (d, J=9 Hz, 2H), 7.43 (d, J=7 Hz, 2H), 7.40-7.23 (m, 11H), 7.03-6.98 (m, 1H), 6.96 (d, J=11 Hz, 2H), 6.65 (d, J=9 Hz, 1H), 5.89 (dd, J=9 Hz, J=5 Hz, 1H), 5.26 (d, J=13 Hz, 1H), 5.04-4.95 (m, 2H), 3.84 (s, 2H), 3.52 (AB system, Δμ=55 Hz, J=19 Hz, 2H).

Spectral data are in accordance with literature values (S. Desgranges, C. C. Ruddle, L. P. Burke, T. M. McFadden, J. E. O'Brien, D. Fitzgerald-Hughes, H. Humphreys, T. P. Smyth, M. Devocelle. β-Lactam-host defence peptide conjugates as antibiotic prodrug candidates targeting resistant bacteria. RSC Advances 2012, 2, 2480).

A solution of 11 (100 mg, 0.146 mmol, 1.0 eq.) in DCM (2 mL) is treated with primary amine 4 (25 mg, 0.160 mmol, 1.2 eq.)), and the stirred mixture cooled in an ice-bath before addition of DIPEA (127 μL, 0.729 mmol, 5.0 eq.). After 5 min, the ice bath is removed and the solution stirred at 30° C. overnight. The mixture is then washed using saturated Na₂CO₃ (2 times) and NaHCO₃ (2 times) aqueous solutions, dried over Na₂SO₄, filtered, and evaporated under reduced pressure. The resulting crude oil is purified via column chromatography on silica gel (DCM/MeOH, 99/1 v/v) to furnish the desired carbamate 12 as a yellow oil (39 mg, 0.056 mmol, 38%).

¹H -NMR (300 MHz, CDCl₃): δ (ppm)=7.57-7.48 (m, 1H), 7.42-7.22 (m, 11H), 7.09-6.84 (m, 3H), 5.94-5.75 (m, 1H), 5.25-4.94 (m, 3H), 3.94-3.63 (m, 2H), 3.49-3.12 (m, 4H), 3.04-2.74 (m, 2H), 2.53-1.98 (m, 4H), 1.81-1.37 (m, 6H).

ESI : [M+H]⁺ m/z found 701.2, calc. 701.2.

To a solution of 12 (39 mg, 0.056 mmol, 1.0 eq.) in dry DCM (1.5 mL) is added 1,3-dimethylbarbituric acid (DMBA) (43 mg, 0.278 mmol, 5.0 eq.), the resulting mixture is degassed using an argon flux, before being treated with tetrakis(triphenylphosphine)palladium(0) Pd(PPh₃)₄ (1 mg, 0.0006 mmol, 0.01 eq.). After completion of the reaction (typically around 4 h), the reaction mixture is evaporated to dryness and purified via chromatography on silica gel (DCM/MeOH, 99/1 v/v) to furnish the desired secondary amine 13 as a yellow oil (15 mg, 0.023 mmol, 41%).

¹H -NMR (300 MHz, CDCl₃): δ(ppm)=7.46-7.17 (m, 10H), 7.04-6.84 (m, 2H), 5.53-5.32 (m, 1H), 5.08-4.92 (m, 1H), 4.30-4.03 (m, 1H), 4.00-3.88 (m, 2H), 3.79-3.71 (m, 2H), 3.55-3.40 (m, 2H), 3.40-2.85 (m, 4H), 2.85-2.59 (m, 1H), 1.88-1.45 (m, 6H).

ESI: [M+H]⁺ m/z found 661.2, calc. 661.2.

To an ice-cold suspension of ELF-97 (7 mg, 0.023 mmol, 1.0 eq.) in dry DCM (1 mL) under an argon atmosphere is added dropwise N,N-diisopropylethylamine (DIPEA) (20 μL, 0.113 mmol, 5.0 eq.), followed by a solution of triphosgene (20 mg, 0.068 mmol, 3.0 eq.) in dry DCM (1 mL). The mixture is stirred for 1 h at 0° C. and overnight at r.t. Next morning, it is reduced to dryness under reduced pressure while the volatiles are trapped in a liquid-nitrogen trap. The latter's contents are subsequently destroyed by addition of ethanolic sodium hydroxyde). The resulting chloroformate of ELF-97 (solid residue) is used without further purification in the next step.

To an ice-cold suspension of the above-prepared chloroformate of ELF-97 (1.0 eq.) in dry DCM (1 mL) and under argon is added dropwise a clear solution of the secondary amine 13 (15 mg, 0.023 mmol, 1.0 eq.). Stirring is continued for another 30 min at 0° C. and then at r.t. overnight. The reaction mixture is washed three times with saturated NaHCO₃ and the organic phase dried over Na₂SO₄, filtered and evaporated under reduced pressure. The crude product is purified via column chromatography on silica gel (PE/EtOAc, 8/2 v/v) to furnish the desired protected probe 14 as an off-white solid (12 mg, 0.012 mmol, 53%).

ESI: [M+H]⁺m/z found 992.0, calc. 992.3.

An ice-cold solution of 14 (12 mg, 0.012 mmol, 1.0 eq.) in dry DCM (1 mL) is treated dropwise with TFA (500 μL, excess) and anisole (7.2 μg, 0.66 mmol, 5.5 eq). The stirred mixture is allowed to warm to r.t., and monitored by mass spectrometry to determine the point of completion (1-2 hours). All volatiles are the removed under reduced pressure. The crude residue is subjected to purification by prep. HPLC (ACN/H₂O 0/100 to 50/50 v/v) to furnish the target compound 15 as a white powder after freeze-drying (1.5 mg, 0.0018 mmol, 15%).

ESI: [M+H]⁺ m/z found 826.3, calc. 826.1.

### Example 2: Detection of Fluorescence of Compound 15

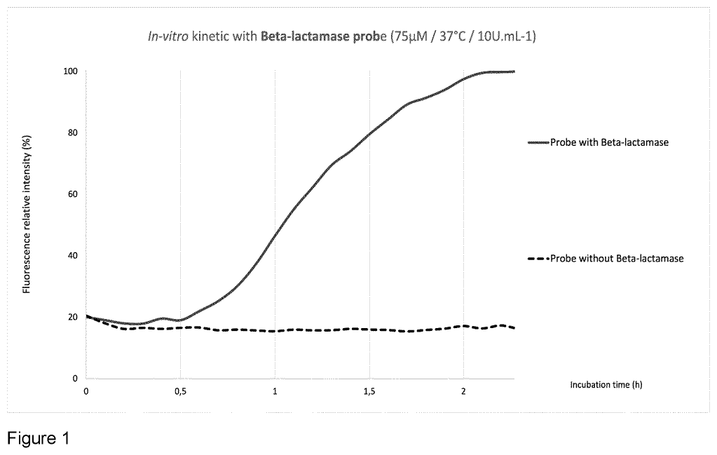

The fluorescence of compound 15 was evaluated with and without a β-lactamase. The test was performed by incubation/chemical reaction in a microwell plate (75 μ, 37° C., 10 U.mL⁻¹). The fluorescence was measured over time by a plate fluorimeter. The obtained results are shown in FIG. 1.

The results show that the compounds according to the invention can detect β-lactamase activity by generating fluorescence (fluorogenic probes). In the presence of β-lactamase, compound 15 is hydrolyzed which leads to the fragmentation of the compound, and the release of a small fluorescent molecule (ELF 97) which generates intense fluorescence. In the absence of the enzyme activity however, no change in fluorescence is observed over 2 hours, thus proving the stability of probe 15 in the incubation medium (physiological).

### Example 3: Synthesis of Compound 25

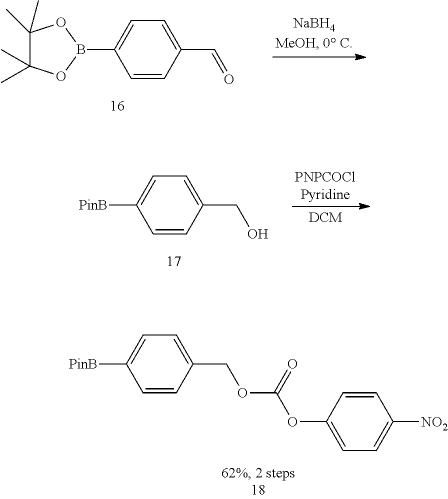

To an ice-cold solution of aldehyde 16 (1 g, 4.3 mmol) in methanol (20 mL) was added sodium borohydride (1 eq, 4.3 mmol, 164 mg). The solution was stirred at 0° C. for 20 minutes before acetone (2 mL) was added. The volatiles were removed under reduced pressure and the slide residue was dissolved in ethyl acetate/water (50 mL/20 mL). The mixture was transferred into a separatory funnel, the aquous phase removed and the organic phase was washed with brine, dried over sodium sulfate and filtered, yielding the crude alcohol 17 in essentially pure form as a yellow pale solid.

The crude alcohol 17 was dissolved in anydrous DCM (20 mL) and p-nitrophenyl chloroformate (1.05 equiv., 912 mg) was added. The flask was placed in an ice bath and pyridine (2 equiv., 8.6 mmol, 0.7 mL) was added dropwise. The reaction was then stirred at room temperature for 16h, and then diluted with diethyl ether and filtered on celite. To the resulting solution was added Celite (20 g) and the solvents were removed under reduced pressure. The celite adsorbed crude mixture was subjected to flash chromatography on silica gel (Petroleum ether/ethyl acetate 8:2) to give the activated carbonate 18 (1.06 g, 2.67 mmol, 62% over 2 steps) as a yellow pale solid.

¹H -NMR (300 MHz, CDCl₃): δ (ppm)=8.35-8.16 (m, 2H), 7.85 (d, J=8.1 Hz, 2H), 7.43 (d, J=8.1 Hz, 2H), 7.41-7.33 (m, 2H), 5.31 (s, 2H), 1.35 (s, 12H).

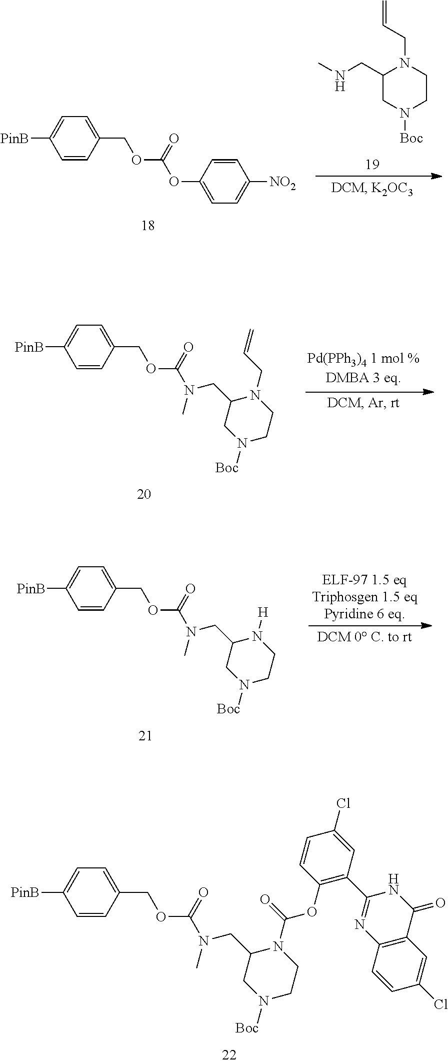

To a solution of N-methyl-N′-allyl-aminomethylpiperidine 19 (1.0 equiv, 0.89 mmol, 240mg) in anhydrous dichloromethane (5mL) was added carbonate 19 (1.05 equiv., 0.93 mmol, 373 mg) and potassium carbonate (5 equiv., 4.45 mmol, 615 mg). Upon completion of the reaction as judged by MS (M+H⁺20=530.4), the reaction mixture was diluted with a 1:1 mixture of petroleum ether and diethyl ether (15 mL), and filtered through celite, the celite rinsed with 100 mL of PE/Et₂O 1:1 mixture. The filtrate was concentrated under reduced pressure to yield essentially pure carbamate 20 as a yellow pale solid, that was used directly in the next step. It could alternatively be purified by flash chromatography on silica gel using Et₂O as eluent for characterization.

20: ¹H -NMR (300 MHz, CDCl₃): δ (ppm)=7.79 (d, J=7.7 Hz, 2H), 7.35 (d, J=8.0 Hz, 2H), 5.94-5.58 (m, 1H), 5.25-4.96 (m, 4H), 3.81-3.02 (m, 8H), 2.97 (s, 3H), 2.70 (brm, 2H), 2.39 (brm, 1H), 1.44 (s, 9H), 1.33 (s, 12H).

Crude 20 was placed in a round bottom flask to which 1,3-dimethylbarbituric acid (3 equiv., 2.67 mmol, 416 mg) was added, followed by DCM (8 mL). The solution was purged with argon for 10 minutes, Pd(PPh₃)₄ (1 mol %, 10 mg) was added, and the mixture stirred at room temperature under argon for 20-60 min. After completion of the reaction as judged by MS, the solvent was evaporated and the crude mixture containing 21 was used without further purification in the next step.

ELF-97 (1.3 equiv, 1.157 mmol, 355 mg) was placed in a round bottom flask under argon, followed by triphosgen (1.3 equiv., 1.157 mmol, 343 mg) and DCM (10 mL). The solution was cooled to 0° C. and pyridine (6 equiv., 0.43 mL) was added dropwise. The ice bath was removed and the solution stirred at rt for 20 min. Volatiles were removed under reduced pressure, DCM (5 mL) was added, and volatiles were removed again under reduced pressure. The solid ELF chlorformate obtained was suspended in DCM (5 mL), the flask cooled in an ice bath, and the crude product 21 (1 equiv., 0.89 mmol) was added in solution in DCM (10 mL), followed by DIPEA (3 equiv., 0.47mL). The reaction was stirred at room temperature for 3 hours until no more 21 was detected in MS. The reaction was then diluted in Et₂O and cooled to 0° C. before sat. Aqueous NaHCO₃ (10 mL) was added. The mixture was transferred in a separatory funnel, the organic phase washed successively with water and brine, dried over Na₂SO₄, filtered and concentrated under educed pressure. The solid residue was partially dissolved in Et₂O and filtered through a pad of silica pretreated with 2.5 w/w % of triethlamine to remove the excess of unreacted ELF-97, rinsing the silica with Et₂O (200 mL). The solution was then concentrated under reduced pressure and subjected to flash chromatography on silica gel (Eluent DCM/Et₂O 1:0 to 3:7) to give pure boronate 22 as a glassy yellow pale solid (536.6 mg, 0.65 mmol, 73%) (M+H⁺22=822.4).

22: ¹H -NMR (300 MHz, CDCl₃): δ (ppm)=8.27-8.20 (m, 1H), 8.01-7.88 (m, 1H), 7.76 (appt, J=7.5 Hz, 1H), 7.70 (d, J=1.9 Hz, 2H), 7.39-7.26 (m, 4H), 7.18-7.01 (m, 1H), 5.23-4.76 (m, 2H), 4.70-4.37 (m, 1H), 4.20-3.81 (m, 3H), 3.70 (m, 1H), 3.38-3.11 (m, 2H), 3.13-2.68 (m, 6H), 1.46 (m, 9H), 1.39-1.26 (m, 12H).

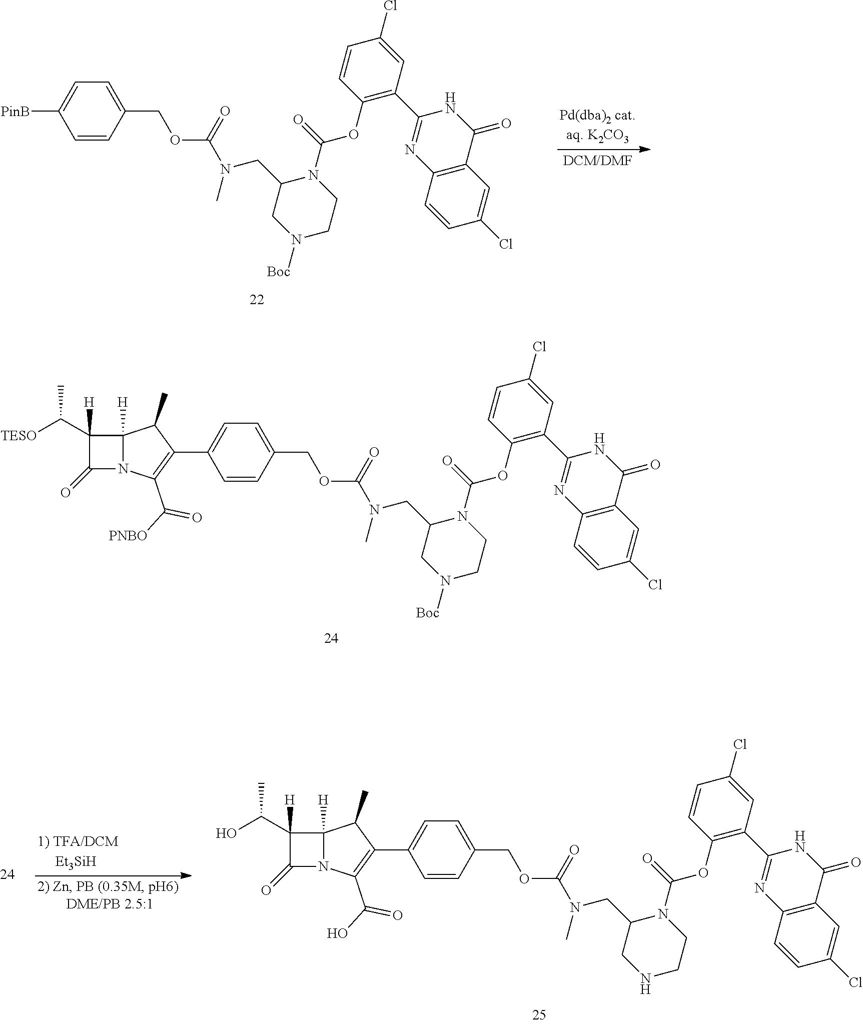

Compound 22 can be coupled with enol triflate 23 using conditions published (Chem. Eur. J. 2020, 26, 3647-3652) for the coupling of Aryl-pinacol-boronate esters with intermediate 23, giving Compound 24, that can be deprotected in conditions described in the above reference to give compound 25.

### Example 4: Synthesis of Compound 32

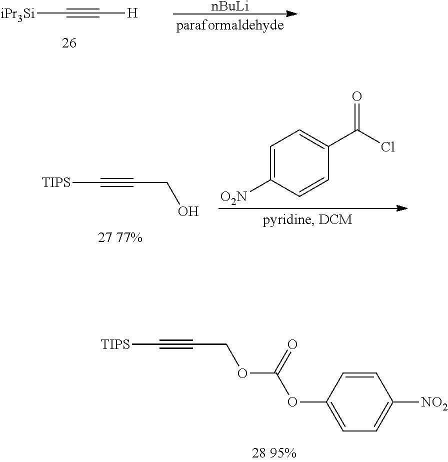

Trisopropylacetylene 26 (1 equiv., 20 mmol, 3.64 g) was placed in a dry round bottom flask under argon and dissolved in anhydrous THF (40 mL). The flask was placed at −78° C. and n-BuLi (1.5 equiv, 30 mmol) was added dropwise over minutes, before the flask was placed at 0° C. for 30 minutes. Paraformaldehyde (e quiv., 3 g) was then added in one portion and the reaction stirred at room temperature for 14 h. The mixture was cooled to 0° C. and aqueous sat. NH₄Cl was added. The mixture was extracted with Et₂O, washed with water, brine, dried over Na₂SO₄ and concentrated under educed pressure. The crude oil was subjected to flash chromatography on silica gel (EP/Et₂O 1:0 to 1:1) to give pure alcohol 27 (3.3 g, 15.5 mmol, 77%) as a colorless oil.

27: ¹H -NMR (300 MHz, CDCl₃): δ (ppm)=4.30 (d, J=5.6 Hz, 2H), 1.07 (s, 21H).

Alcohol 27 (3.3 g, 15.5 mmol, 1 equiv.) was dissolved in anydrous DCM (30mL) and p-nitrophenyl chloroformate (1.05 equiv., 3.29 g) was added. The flask was placed in an ice bath and pyridine (2.5 equiv., 38.75 mmol, 3.2 mL) was added dropwise. The reaction was then stirred at room temperature for 16 h, and then diluted with diethyl ether and filtered on celite, rinsing with Et₂O. The solvents were removed under reduced pressure and the crude mixture was subjected to flash chromatography on silica gel (eluent EP/CHCl₃ 1:0 to 0:1) to give activated carbonate 28 (5.56 g, 14.7 mmol, 95%) as a colorless oil.

28: ¹H -NMR (300 MHz, CDCl₃): δ (ppm)=8.42-8.18 (m, 2H), 7.45-7.34 (m, 2H), 4.85 (d, J=5.4 Hz, 2H), 1.07 (s, 21H).

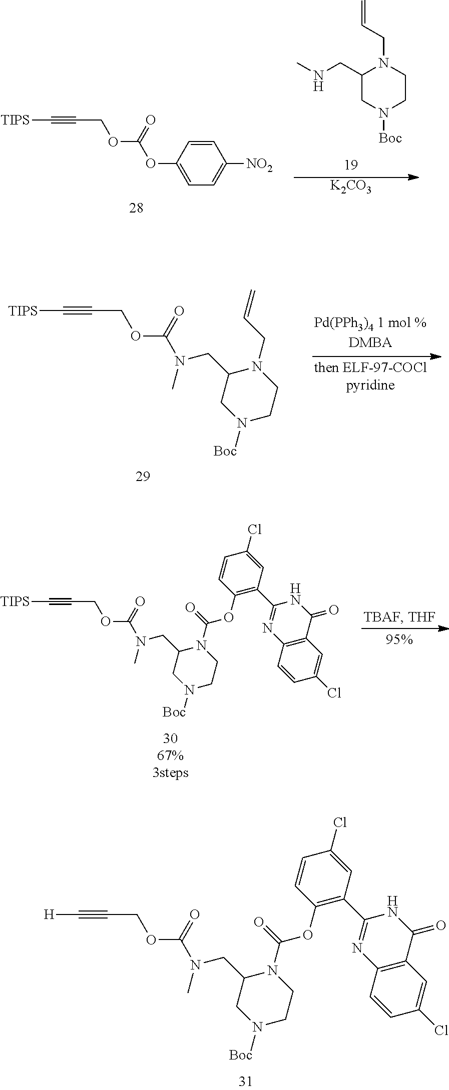

To a solution of N-methyl-N′-allyl-aminomethylpiperidine 19 (1.0 equiv, 0.868 mmol, 233 mg) in anhydrous dichloromethane (5 mL) was added carbonate 28 (1.1 equiv., 0.96 mmol, 362 mg) and potassium carbonate (5 equiv., 4.4 mmol, 621 mg). Upon completion of the reaction as judged by MS (M+H⁺29=508.7), the reaction mixture was diluted with a 1:1 mixture of petroleum ether and diethyl ether (15 mL), and filtered on celite, the celite rinsed with 100 mL of PE/Et2O 1:1 mixture. The filtrate was concentrated under reduced pressure to yield essentially pure carbamate 29 as a yellow pale oil, that was used directly in the next step.

The crude carbamate 29 was placed in a round bottom flask to which 1,3-dimethylbarbituric acid (3 equiv., 2.6 mmol, 406 mg) was added, followed by DCM (8mL). The solution was purged with argon for 10 minutes, Pd(PPh₃)₄ (1 mol %, 10 mg) was added, and the mixture stirred at room temperature under argon for 20-60 min. In parallel, ELF chlorofrmate was preapared as for example 3, by reaction of ELF-97 (1.3 equiv., 1.13 mmol, 347 mg) with triphosgen (1.3 equiv., 1.13 mmol, 335 mg) and pyridine (6 equiv., 5 mmol, 0.41 mL), successive evaporation/dissolution in DCM, before being placed in DCM (10 mL) in an ice-cold bath. After completion of the deallylation reaction, as judged by MS, the solution containing deallylated 29 was cannulated on the cold solution of ELF chloroformate in DCM and the flask rinsed twice with DCM (2+2 mL). The reaction was stirred at room temperature for 14 h and placed in an ice bath, diluted with Et₂O (50 mL) and sat aqueous NaHCO₃ (20 mL) was added. 30 was extracted with Et₂O, the organic phase washed with water, brine, dried over Na₂SO₄, filtered and concentrated under reduce pressure. Purification of the crude mixture as for example 3, by a first filtration on a pad of Et₃N-impregnated silica followed by flash chromatography over silicagel (DCM/Et₂O 0:1 to 1:0), afforded pure 30 as a glassy colorless solid (456 mg, 0.57 mmol, 67%, 3 steps). (M+H⁺30=799.3).

30: ¹H -NMR (300 MHz, CDCl₃): δ (ppm)=8.32-8.17 (m, 1H), 8.02 (d, J=2.7 Hz, 1H), 7.72 (pseudoq, J=2.5, 2.1 Hz, 2H), 7.56-7.45 (m, 1H), 7.25-7.08 (m, 1H), 4.79-4.29 (m, 3H), 4.26-3.58 (m, 5H), 3.36-2.70 (m, 7H), 1.48 (s, 9H), 1.05 (s, 21H).

Pure 30 (0.47 mmol, 376 mg) was placed in a flask, dissolved in technical-grade THF (15 mL), and placed in an ice bath. A solution of TBAF (1 M in THF, 1. 02 equiv., 479 μL) was then added dropwise and the reaction stirred 16h at room temperature. The flask was placed in an ice bath and aqueous sat. NaHCO₃ (10 mL) was added dropwise. 31 was extracted with Et₂O, washed with water, brine, dried over Na₂SO₄, filtered and concentrated under reduce pressure. Purification of the crude mixture flash chromatography over silicagel (DCM/Et₂O 0:1 to 1:0), afforded pure 31 as a glassy colorless solid (294 mg, 0.46 mmol, 95%). (M+H⁺31=644.3).

31: ¹H -NMR (300 MHz, CDCl₃): δ (ppm)=¹H NMR (300 MHz, Chloroform-d) δ 8.28-8.16 (m, 1H), 8.02-7.89 (m, 1H), 7.80-7.63 (m, 2H), 7.54-7.43 (m, 1H), 7.25-7.12 (m, 1H), 4.87-4.35 (m, 3H), 4.33-3.54 (m, 4H), 3.31-3.10 (m, 1H), 3.10-2.68 (m, 6H), 2.46-2.22 (m, 1H), 1.47 (s, 9H).

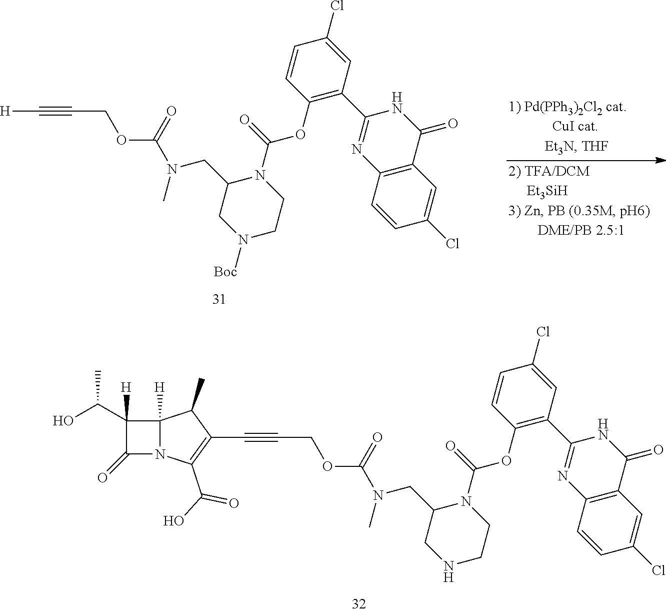 [SMILES: C#CCOC(=O)N(C)CC1CN(C(=O)OC(C)(C)C)CCN1C(=O)Oc1ccc(Cl)cc1-c1nc2ccc(Cl)cc2c(=O)[nH]1 ; C[C@@H](O)[C@H]1C(=O)N2C(C(=O)O)=C(C#CCOC(=O)N(C)CC3CNCCN3C(=O)Oc3ccc(Cl)cc3-c3nc4ccc(Cl)cc4c(=O)[nH]3)[C@H](C)[C@H]12]

Compound 31 can undergo Sonogashira coupling using Pd/Cu co-catalysis to give an coupled intermediate that can be deprotected as for example 3 to give compound 32.

1. A compound of formula (I): 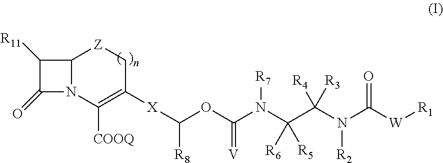
in which:
W is —O— or —NR₁₃—, with R₁₃ being a C₁-C₄ alkyl or a hydrogen atom;
R₁ is such that HWR₁, obtained after cleavage of the —C(O)—WR₁ bond present in formula (I), belongs to the class of fluorophores;
R₂, R₃ and R₄ are defined as follows:
either R₂ is a C₁-C₄ alkyl, R₃ is a C₁-C₄ alkyl or a hydrogen atom, and R₄ is a C₁-C₄ alkyl;
or R₃ is a C₁-C₄ alkyl or a hydrogen atom and R₂ and R₄ are bonded to each other and form a —(CH₂)ₚ—Y_{q}—(CH₂)ᵣ— chain in direction of R₂ toward R₄, wherein
Y is O, NR₁₄, N(R₁₄)₂⁺or S,
p=0, 1, 2, 3, 4 or 5,
q=0 or 1,
r=0, 1, 2, 3, 4 or 5,
p+q+r=3,4,5, or 6,and
each R₁₄ represents independently a hydrogen atom, a C₁-C₆ alkyl, an amino protecting group, or -(L)_{q}-GP, with q which is equal to 0 or 1, L which is a linking arm and GP which is a hydro solubilizing group;
or R₂ is a C₁-C₄ alkyl and R₃ and R₄ are bonded together and form, with the carbon atom to which they are bonded, an aliphatic carbocycle;
R₅ and R₆ are identical or different and represent, independently of each other, a hydrogen atom, a C₁-C₄ alkyl, or a C₅-C₁₀ aryl;
R₇ is a hydrogen atom, or a group selected from C₁-C₄ alkyl and C₁-C₄ alkoxy;
R₈ represents a hydrogen atom;
V represents an oxygen atom or a sulfur atom;
n is 0 or 1;
Z is —S—, —SO—or —CR₉R₁₀—; with R₉ and R₁₀ being identical or different and representing, independently of each other, a hydrogen atom or a C₁-C₄ alkyl;
Q is H, a cation or R16, where R16 is selected from C₁-C₆ alkyl optionally substituted with an aryl or a —O(CO)—R, with R being independently selected from H, C₁-C₆ alkyl and C₃-C₆ cycloalkyl; and
R₁₁ is an organyl group, an organylamino group, an organyloxy group, or an organylthio group,
X represents a bond, or a group selected from

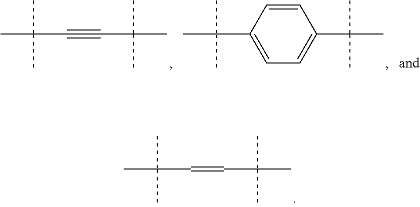

2. Compound (I) according to claim 1 of formula (Ia): 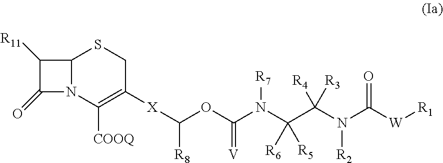
where R₁, R₂, R₃, R₄, R₅, R₆, R₇, R₈, R₁₁, Q, W, X and V are as defined in claim 1.

3. Compound (I) according to claim 1 of formula (Ib): 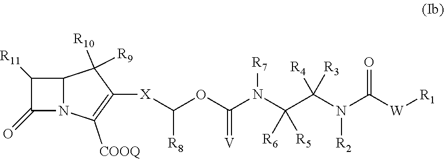
where R₁, R₂, R₃, R₄, R₅, R₆, R₇, R₈, R₉, R₁₀, R₁₁, Q, W, X and V are as defined in claim 1.

4. Compound (I) according to claim 1, wherein R₁ comprises an aromatic group comprising one or more aromatic rings, optionally substituted, which rings can comprise one or more hetero-atoms chosen from among the nitrogen, oxygen or sulfur atoms and/or one or more carbon atoms in the form of a C═O carbonyl.

5. Compound (I) according to claim 1, wherein WRᵢ is an aromatic group —OR₁ according to formula (A1): 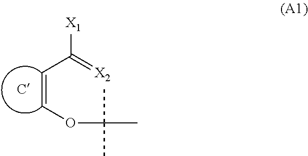
in which:
either X₂ is an oxygen atom and X₁ is a —NH₂, —OH, —SH, C₁-C₂₀ alkyl, C₅-C₂₄ aryl, C₂-C₆ alkenyl, —O-(C₁-C₂₀ alkyl), —O-phenyl, —NH-(C₁-C₂₀ alkyl), —NH-phenyl, —S-(C₁-C₂₀ alkyl) or —S-(C₅-C₂₄ aryl group), said alkyl, aryl, alkenyl and phenyl groups being optionally substituted;
or X₂ represents a nitrogen atom and is bound to X₁ which then represents CH, O, S, N or, NH to form a C₅-C₂₄ heteroaryl, optionally substituted; 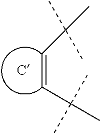
represents a C₅-C₂₄ aryl or a C₅-C₂₄ heteroaryl, optionally substituted, 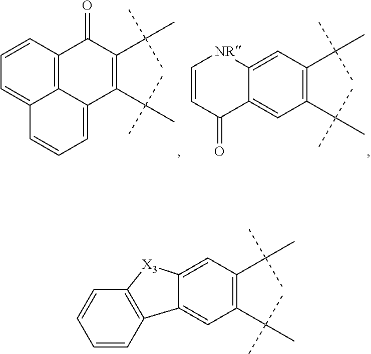
or —OR₁ is of the aryloxy type and corresponds to one of the following structures (A2), (A3) or (A4): 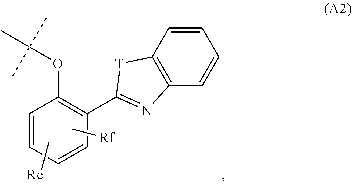
in which:
T is —NH—C(O)—, —S—, —O—, —NH—, —N(C₁-C₂₀ alkyl)- or —N(C₅-C₂₄ aryl)-;
Re is a hydrogen atom or an electron-withdrawing group such as —CN or —COORh, with Rh which represents a C₁-C₄ alkyl group, or Re is —CONRiRj, with Ri and Rj, identical or different, which represent a hydrogen atom, or a C₁-C₄ alkyl group, or Re is —CF₃, a C₂-C₆ alkenyl, or a heteroaryl, said heteroaryl and alkenyl being optionally substituted;
Rf is a hydrogen atom, a chlorine, bromine, iodine or fluorine atom, —OH, —NH₂, —NRkRI, —NHRk or —ORk, with Rk and RI, identical or different, which each, independently, represent a C₁-C₄ alkyl group;
or Re and Rf are bonded to each other to form a hydrocarbon chain comprising 4 or 5 members, saturated or unsaturated, substituted or non-substituted, possibly interrupted by one or more hetero-atoms chosen from among N, S and O; and
Rg is a hydrogen, Br, Cl, I or F atom; 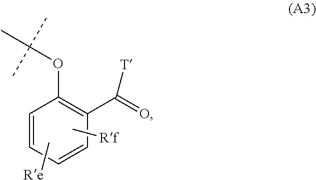
in which:
T′ is —NH₂, —OH, a C₅-C₂₄ aryl group, a C₁-C₄ alkyl group, —SH, —NHR′g, —OR′g, —NR′gRh', —SR′g, or an optionally substituted C₂-C₆ alkenyl group, or a heteroaryl, R′g and Rh′, identical or different, representing a C₁-C₄ alkyl or C₅-C₂₄ aryl group;
R′e is a hydrogen atom or an electron-withdrawing group such as —CN or —COOR′i, with R′i which represents a C₁-C₄ alkyl group, or R′e is —CONR′jR′k, with R′j and R′k, identical or different, which represent a hydrogen atom, or a C₁-C₄ alkyl group, or R′e is —CF₃, or a 2-oxazolyl, 2-thiazolyl, 2-imidazolyl, 2-benzo imidazolyl, 4-pyrimidinone-2-yl or quinazolinone-2-yl group;
R′f is a hydrogen, chlorine, bromine, iodine or fluoride atom, —OH, —NH₂, -NR′IR′m or —OR′I, with R′I and R′m, identical or different, which represent a C₁-C₄ alkyl group;
or R′e and R′f are bonded to each other to form a hydrocarbon chain comprising 4 or 5 members, saturated or unsaturated, substituted or non-substituted, possibly interrupted by one or more hetero-atoms chosen from among N, S and O 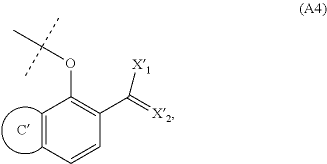
in which
either X′₂ is an oxygen atom and X′₁ is a —NH₂, —OH, —SH, C₁-C₂₀ alkyl, C₅-C₂₄ aryl, C₂-C₆ alkenyl, —O-(C₁-C₂₀ alkyl), —O-phenyl, —NH-(C₁-C₂₀ alkyl), —NH-phenyl, —S-(C₁-C₂₀ alkyl) or —S-(C₅-C₂₄ aryl group), said alkyl, aryl, alkenyl and phenyl groups being optionally substituted;
or X′₂ represents a nitrogen atom and is bound to Xᵢ which then represents CH, O, S, N or, NH to form a C₅-C₂₄ heteroaryl, optionally substituted; 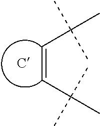
represents a C₅-C₁₀ aryl or a C₅-C₁₀ heteroaryl, optionally substituted.

6. Compound (I) according to claim 1, wherein R1 is selected from the group consisting of fluoresceins (including rhodamines and rhodols), coumarins (including 7-amino- and 7-hydroxy-coumarins), cyanines, phenoxazines and acridinones.

7. Compound (I) according to claim 1, wherein R₁₁ is selected from C₁-C₆ alkyl, C₁-C₆ heteroalkyl, C₃-C₆ cycloalkyl, C₁-C₆ haloalkyl, C₂-C₆ alkenyl, C₂-C₆ alkynyl, heterocyclyl having 5 to 10 ring atoms, C₅-C₁₀ aryl, heteroaryl having 5 to 10 ring atoms, C₇-C₁₆ aralkyl, and NR″R″′;
said alkyl, cycloalkyl, hetroalkyl, haloalkyl, alkenyl, alkynyl, heterocyclyl, aryl, heteroaryl and aralkyl being optionally substituted with one or more substituents independently selected from oxo, halogen, C₁-C₆ alkyl, C₁-C₆ heteroalkyl, C₃-C₆ cycloalkyl, C₁-C₆ haloalkyl, heterocyclyl having 5 to 10 ring atoms, C₅-C₁₀ aryl, heteroaryl having 5 to 10 ring atoms, —OH, —NR″R″′, —NO₂, —CN, —O—(CO)—R and —(CO)—R;
with each R being independently selected from H, C₁-C₆ alkyl, C₁-C₆ alkoxy and —NR″R′′; and
with each R″ and R″′ being independently selected from H and C₁-C₆ alkyl.

8. Compound (I) according to claim 1 of formula (Ic): 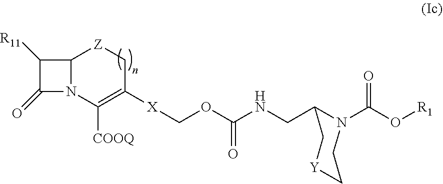
where R₁, R₁₁, Z, Q, X and n are as defined in claim 1, and Y represents —CH₂—, —NR₁₄—, or —N(R₁₄)₂⁺—, with each R₁₄ represents independently a hydrogen atom, a C₁-C₆ alkyl, an amino protecting group or -(L)_{q}-GP, with q which is equal to 0 or 1, L which is a linking arm and GP which is a hydro solubilizing group.

9. Compound (I) according to claim 1 of formula (Id): 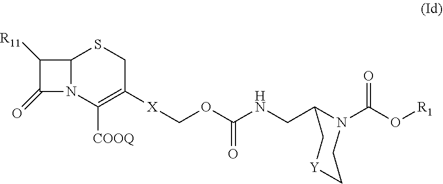
where R₁, R₁₁, X and Q are as defined in claim 1, and Y represents —CH₂—, —NR₁₄—, or —N(R₁₄)₂⁺—, with each R₁₄ represents independently a hydrogen atom, a C₁-C₆ alkyl, an amino protecting group or -(L)_{q}-GP, with q which is equal to 0 or 1, L which is a linking arm and GP which is a hydro solubilizing group.

10. Compound (I) according to claim 1 of formula (Ie): 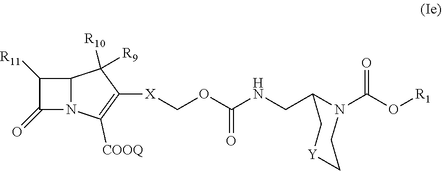
where R₁, R₉, R₁₀, R₁₁, X and Q are as defined in claim 1, and Y represents —CH₂—, —NR₁₄—, or —N(R₁₄)₂⁺—, with each R₁₄ represents independently a hydrogen atom, a C₁-C₆ alkyl, an amino protecting group or -(L)_{q}-GP, with q which is equal to 0 or 1, L which is a linking arm and GP which is a hydro solubilizing group.

11. Method for the in vitro or ex vivo detection of a β-lactamase, comprising the steps of:
putting a sample to be analyzed into contact with a compound (I) according to claim 1,
applying suitable conditions in order to make possible the formation of a fluorescent precipitate by cleavage of the covalent bond between C(═V) and NR₇, followed by a cleavage of the —C(O)—WR₁, bond, leading to the release of HWR₁,
quantitative or qualitative analysis of the fluorescent precipitate, and
correlate the quantitative or qualitative analysis of the fluorescent precipitate to the presence or absence of β-lactamase in the sample.

12. Method for the in vitro or ex vivo detection of antibiotic-resistant bacteria comprising the steps of:
putting a sample to be analyzed into contact with a compound (I) according to claim 1,
applying suitable conditions in order to make possible the formation of a fluorescent precipitate by cleavage of the covalent bond between C(═V) and NR₇, followed by a cleavage of the —C(O)—WR₁, bond, leading to the release of HWR₁,
quantitative or qualitative analysis of the fluorescent precipitate, and
correlate the quantitative or qualitative analysis of the fluorescent precipitate to the presence or absence of antibiotic-resistant bacteria in the sample.

13. A kit for detecting a β-lactamase, said kit comprising a compound according to claim 1.

14. A device for detecting a β-lactamase, said device comprising a compound according to claim 1.

15. A method for the in vivo detection, in animals or in human beings, of a β-lactamase, using a compound (I) according to claim 1.

16. A compound of formula (II): 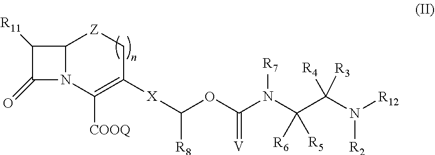
in which:
R₂, R₃, R₄, R₅, R₆, R₇, R₈, R₁₁, Z, n, Q, X and V are as defined in claim 1,
R₁₂ represents a hydrogen atom, or an amine function protecting group.

17. Process for the preparation of a compound of formula (I) according to claim 1 comprising the following steps:
implementation of a compound (II), 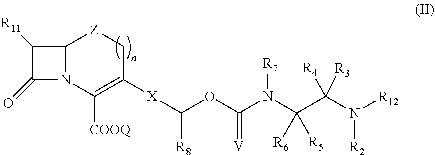
in which:
R₂, R₃, R₄, R₅, R₆, R₇, R₈, R₁₁, Z, n, Q, X and V are as defined in claim 1, where R₁₂ represents a hydrogen atom;
implementation of a compound (III) of formula 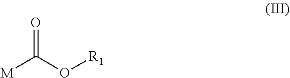
with R₁ as defined in claim 1 and M representing a leaving group,
obtaining compound (I) by addition reaction of said compound (II) to compound (III).

18. Process for the preparation of a compound of formula (I) according to claim 1, where X is 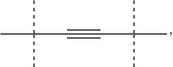
comprising the following steps:
implementation of a compound (VII) of the following formula: 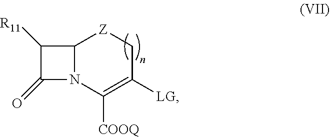
implementation of a compound (VIII) of formula 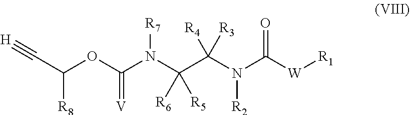
and obtaining compound (I) by reaction of said compound (VII) with compound (VIII),
where R₁, R₂, R₃, R₄, R₅, R₆, R₇, R₈, V, R₁₁, Q, W, Z and n are as defined in claim 1, and LG represents a leaving group.

19. Process for the preparation of a compound of formula (I) according to claim 1, where X is 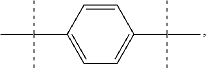
comprising the following steps:
implementation of a compound (VII) of the following formula: 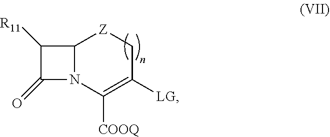
implementation of a compound (X) of formula 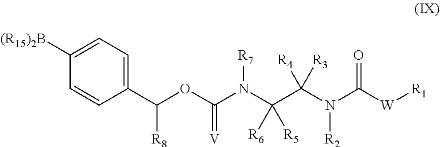
and obtaining compound (I) by reaction of said compound (VII) with compound (IX),
where R₁, R₂, R₃, R₄, R₅, R₆, R₇, R₈, V, R₁₁, Q, W, Z and n are as defined in claim 1, LG represents a leaving group, and R₁₅ represents OH, or both R₁₅ are bonded to each other and forms, together with the B atom to which they are bonded, a heterocycle having from 5 to 10 ring atoms.

20. Process for the preparation of a compound of formula (I) according to claim 1, where X is 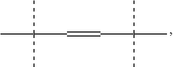
comprising the following steps:
implementation of a compound (VII) of the following formula 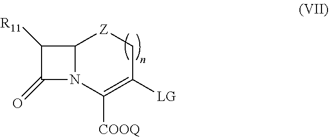
implementation of a compound (X) of formula 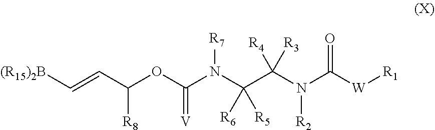
and obtaining compound (I) by reaction of said compound (VII) with compound (X),
where R₁, R₂, R₃, R₄, R₅, R₆, R₇, R₈, V, R₁₁, Q, W, Z and n are as defined in claim 1, LG represents a leaving group, and R₁₅ represents OH, or both R₁₅ are bonded to each other and forms, together with the B atom to which they are bonded, a heterocycle having from 5 to 10 ring atoms.
